(19) United States
(12) Patent Application Publication
(10) Pub. No.: US 2019/0183991 A1
(43) Pub. Date: Jun. 20, 2019

(54) METHOD OF PREVENTING ACUTE ATTACKS OF HEREDITARY ANGIOEDEMA ASSOCIATED WITH C1 ESTERASE INHIBITOR DEFICIENCY

(71) Applicant: CSL BEHRING GMBH, Marburg (DE)

(72) Inventors: Thomas MACHNIG, Ingelheim (DE); Dipti PAWASKAR, Philadelphia, PA (US); Michael TORTORICI, Berwyn, PA (US); Ingo PRAGST, Edertal (DE); Ying ZHANG, Chesterbrook, PA (US)

(21) Appl. No.: 16/327,553

(22) PCT Filed: Aug. 23, 2017
(86) PCT No.: PCT/EP2017/071237

§ 371 Date: Feb. 22, 2019

Related U.S. Application Data
(60) Provisional application No. 62/378,299, filed on Aug. 23, 2016.
(60) Provisional application No. 62/420,776, filed on Nov. 11, 2016.

(51) Int. Cl. A61K 38/57; A61P 7/10; A61K 9/00; G01N 33/68; G16H 20/10

(52) U.S. Cl. CPC A61K 38/57; A61P 7/10; G16H 20/10; G01N 33/6893; A61K 9/0019

(57) ABSTRACT

The invention relates to a method for determining a dosing scheme for the treatment of hereditary angioedema and/or the prevention of hereditary angioedema attacks with C1 esterase inhibitor to optimize treatment response in an individual patient. Accordingly, the present invention provides means for determining individual C1 esterase inhibitor dosing schemes that result in an optimal treatment/prevention outcome.

### TECHNICAL FIELD

The invention relates to a method for determining a dosing scheme for the treatment of hereditary angioedema and/or the prevention of hereditary angioedema attacks with C1 esterase inhibitor to optimize treatment response in an individual patient. Accordingly, the present invention provides means for determining individual C1 esterase inhibitor dosing schemes that result in an optimal treatment/prevention outcome.

### BACKGROUND

C1 esterase inhibitor (C1-INH), a plasma glycoprotein with a molecular weight of 104 kDa, belongs to the protein family of serine protease inhibitors (serpins), which regulate the activity of serine proteases by inhibiting their catalytic activity (Bock S C, et al., Biochemistry 1986, 25: 4292-4301). C1-INH inhibits the classical pathway of the complement system by inhibiting the activated serine proteases C1s and C1r. Furthermore, C1-INH is a major inhibitor of the contact activation system due to its ability to inhibit the activated serine proteases factor XIIa (FXIIa), factor XIa (FXIa), and plasma kallikrein (Davis A E, Clin. Immunol. 2005, 114: 3-9; Caliezi C et al., Pharmacol. Rev. 2000, 52: 91-112). Deficiency in C1-INH leads to the clinical manifestation of hereditary angioedema (HAE), which is characterized by episodes of acute angioedema attacks in subcutaneous or submucosal tissues such as the skin, larynx, or visceral organs (Longhurst H, et al. Lancet 2012, 379: 474-481) which last between 1 and 7 days and occur at irregular intervals. Abnormalities in C1-INH plasma content or in its functional activity (often referred to as a deficiency of functional C1-INH) result from various large and small mutations in the C1-INH gene (vide supra) (Karnaukhova E, J. Hematol. Thromb. Dis., 2013, 1-7).

Two types of hereditary C1-INH deficiency generally exist. The more prevalent type I HAE is characterized by low content (below 35% of normal) and low inhibitory activity of C1-INH in the circulation. Type II HAE is associated with normal or elevated antigenic levels of C1-INH of low functional activity. Recently, HAE with normal C1-INH (also known as type III HAE) has been described in two subcategories: (1) HAE due to mutation in the factor XII gene and, as a result, increased activity of factor XII leading to a high generation of bradykinin, and (2) HAE of unknown genetic cause. HAE attacks can be treated effectively by administering C1-INH (Longhurst H, et al., Lancet 2012, 379: 474-481; Bork K, Allergy Asthma Clin. Immunol. 2010, 6: 15). Moreover, administration of C1-INH has been shown to prevent edema formation in patients when given prophylactically. C1-INH is currently marketed e.g. as Berinert® (CSL Behring), Cetor® (Sanquin), Cinryze® (Shire), Ruconest®/Rhucin® (recombinant C1 inhibitor by Pharming). Due to its inhibitory effects on the complement and the contact activation systems, C1-INH substitution restores normal homeostatic function and inhibits the excessive formation of vasoactive peptides such as bradykinin, which mediate the formation of angioedema.

Long-term prophylaxis of HAE aims to prevent or to minimize the number and severity of angioedema attacks and ideally prevent any attacks to occur. However, the medications currently available for long-term prophylaxis are in many cases not optimal. Oral antifibrinolytics requiring multiple daily doses are relatively ineffective and frequently associated with significant side effects. Anabolic androgens are convenient to take and usually effective at doses <200 mg/day but can be associated with significant risk of serious side effects. The only approved prophylactic treatment which is most widely used by HAE patients who suffer from frequent and/or severe attacks is long-term replacement therapy with C1-INH preparations.

Several formulations of C1-INH require intravenous access, imposing a burden on the patient and healthcare providers. Since plasma levels of functional C1-INH fall rapidly following intravenous administration of therapeutic dosages of C1-INH concentrates, reaching near basal levels within 3 days, regular, usually twice weekly, infusions are necessary.

Recently, it has been demonstrated that prophylactic treatment of hereditary angioedema with C1-INH replacement therapy can be improved and simplified by subcutaneous administration of a low volume formulation of a C1-INH concentrate (Zuraw et al., Allergy, 2015, DOI:10.1111/a11.12658). While prophylactic C1-INH has been shown effective in reducing the attack rate in most patients, treatment response is highly variable and currently there is no method to determine an optimal dosing strategy for patients who have insufficient treatment response (Zuraw and Kalfus, 2012, The American Journal of Medicine).

Accordingly, the present application fulfills an unmet need in the art by providing means for determining the optimal prophylactic dose of C1-INH for individual patients suffering from hereditary angioedema. The accordingly determined prophylactic dose is optimized for each individual patient resulting in improved treatment response in terms of a maximum reduction or complete prevention of acute hereditary angioedema attacks.

### SUMMARY OF THE INVENTION

Surprisingly, it has been found that, in patients suffering from hereditary angioedema, C1-INH functional activity levels inversely correlate with the risk of experiencing an angioedema attack. This finding contradicts existing views according to which C1-INH activity levels of HAE patients are not predictive for the severity and frequency of angioedema attacks and, except for the diagnosis of HAE, it is not recommended to regularly monitor functional C1-INH activity levels while patients are on C1-INH replacement therapy (e.g., Zuraw et al., J Allergy Clin Immunol: In Practice, Vol 1, Number 5; September/October 2013). The present invention allows improving treatment response in terms of further reducing the risk of experiencing an angioedema attack by adjusting the current C1-INH dosing scheme based on the newly established relationship between C1-inhibitor functional activity and relative risk of an HAE attack. Accordingly, further improvement of the symptomatology is achieved. The present finding allows adjusting and/or selecting the dosing scheme necessary in order to achieve a better treatment response. By implementing the present invention, dosing schemes can be determined and/or improved for individual patients resulting in an optimal treatment response.

In one embodiment, the present invention relates to the provision of a method for determining a C1-INH dosing scheme for individual patients in order to achieve optimal treatment of hereditary angioedema and/or optimal prevention of angioedema attacks. Therefore, an individualized C1-INH dosing scheme for patients is provided. The method for determining a dosing scheme for C1-INH for the treatment of hereditary angioedema and/or the prevention of hereditary angioedema attacks in an individual patient comprises the following steps:
(i) determining baseline C1-INH functional activity (Cr) in a sample obtained from the patient before C1-INH treatment,
(ii) predefining the desired relative risk reduction h(t),
(iii) determining the corresponding target C1-INH functional activity (Cp) based on a model, preferably a model based on formula

$$Cp = \frac{{e}^{3.4} \times ( log ( relative h ( t ) ) + \frac{- 10.5 \times Cr}{{e}^{3.4} + Cr} )}{- 10.5 - log ( relative h ( t ) ) - \frac{- 10.5 \times Cr}{{e}^{3.4} + Cr}}$$

wherein Cr is the baseline value determined in step (i) and relative h(t) is the desired relative risk reduction predefined in step (ii), and
(iv) determining the C1-INH dosing scheme required to maintain the patient's trough level C1-INH functional activity above the target C1-INH functional activity.

The present invention also relates to the provision of a method for adjusting a C1-INH dosing scheme for individual patients in order to achieve optimal treatment of hereditary angioedema and/or optimal prevention of angioedema attacks. Therefore, an individualized C1-INH dosing scheme for patients is provided. The method for adjusting a dosing scheme for C1-INH for the treatment of hereditary angioedema and/or the prevention of hereditary angioedema attacks in an individual patient comprises the following steps:
(i) determining baseline C1-INH functional activity (Cr) in a sample obtained from the patient before C1-INH treatment,
(ii) determining trough C1-INH functional activity in a sample obtained from the patient during ongoing treatment with a standard dose of C1-INH,
(iii) determining the optimal relative risk reduction h(t) based on the patient's treatment response to the treatment of step (ii),
(iv) determining the corresponding target C1-INH functional activity (Cp) based on a model, preferably a model based on formula

$$Cp = \frac{{e}^{3.4} \times ( log ( relative h ( t ) ) + \frac{- 10.5 \times Cr}{{e}^{3.4} + Cr} )}{- 10.5 - log ( relative h ( t ) ) - \frac{- 10.5 \times Cr}{{e}^{3.4} + Cr}}$$

wherein Cr is the baseline value determined in step (i) and relative h(t) is the desired relative risk reduction determined in step (iii), and
(v) determining the C1-INH dosing scheme required to maintain the patient's trough level C1-INH functional activity above the target C1-INH functional activity based on the trough C1-INH functional activity determined in step (ii).

The present invention also relates to the provision of a further method for adjusting a C1-INH dosing scheme for individual patients in order to achieve optimal treatment of hereditary angioedema and/or optimal prevention of angioedema attacks. The method for adjusting a dosing scheme for C1-INH for the treatment of hereditary angioedema and/or the prevention of hereditary angioedema attacks in an individual patient comprises the following steps:
(i) determining trough C1-INH functional activity in a sample obtained from the patient during ongoing treatment with a standard dose of C1-INH,
(ii) determining the optimal risk reduction h(t) based on the patient's treatment response to the treatment of step (i),
(iii) determining the corresponding target C1-INH functional activity (Cp) based on a model, preferably a model based on formula

h(t)=exp(0.08)*(age/42)\hat{ }1.05*exp((−10.5)*Cp/(exp(3.4)+Cp))
wherein h(t) is the risk reduction determined in step (ii), and
(iv) determining the C1-INH dosing scheme required to maintain the patient's trough level C1-INH functional activity above the target C1-INH functional activity (Cp) based on the trough C1-INH functional activity determined in step (i).

The present invention also relates to a method for determining a therapeutic C1-INH concentration (Cp) for the treatment of hereditary angioedema and/or the prevention of hereditary angioedema attacks in an individual patient, using an age-dependent risk-for-an-attack model.

The model may involve the following parameters:
(i) background risk (B0),
(ii) effect of patient age on background risk (Age on B0),
(iii) maximum C1-INH effect (Eₘₐₓ), and
(iv) half maximal effective concentration of C1-INH (EC₅₀).

In one embodiment, the model is based on formula

$$h = {e}^{BO} \times {( \frac{age}{42} )}^{Age on B 0} \times e ( ( E max ) \times \frac{Cp}{( {e}^{EC 50} + Cp )} )$$

wherein h is the risk for an attack and age is the individual patient's age.

Further provided is C1-INH for use in the treatment of hereditary angioedema and/or the prevention of hereditary angioedema attacks, wherein the dosing scheme for C1-INH is determined for an individual patient by the steps of the method for determining a dosing scheme described herein. Also provided is C1-INH for use in the treatment of hereditary angioedema and/or the prevention of hereditary angioedema attacks, wherein the adjustment of the dosing scheme for C1-INH is determined for an individual patient by the steps of the method for adjusting a dosing scheme described herein.

The present invention also relates to a method of treating hereditary angioedema and/or of preventing hereditary angioedema attacks in an individual patient, comprising administering C1-INH to a patient, wherein the dosing scheme for C1-INH is determined by the method for determining a dosing scheme described herein. Further provided is a method of treating hereditary angioedema and/or of preventing hereditary angioedema attacks in an individual patient, comprising administering C1-INH to a patient, wherein the dosing scheme for C1-INH is adjusted by the method for adjusting a dosing scheme described herein.

In one embodiment, the present invention relates to a computer program product stored on a computer usable medium, comprising: computer readable program means for causing a computer to carry out the steps of the method for determining or adjusting a dosing scheme. In a further embodiment, a computer comprising the computer program product stored on a computer usable medium is provided. Also provided is a device for determining/adjusting a dosing scheme for C1-INH for the treatment of hereditary angioedema and/or the prevention of hereditary angioedema attacks in an individual patient comprising: (i) a unit for analyzing C1-INH functional activity in a sample obtained from a patient, and (ii) the computer.

In a further embodiment, the invention relates to a kit comprising (i) a pharmaceutical composition comprising C1-INH, and (ii) instructions for carrying out the method for determining a dosing scheme described herein and/or instructions for using the computer program product described herein. In another embodiment, the invention relates to a kit comprising (i) a pharmaceutical composition comprising C1-INH, and (ii) instructions for carrying out the method for adjusting a dosing scheme described herein and/or instructions for using the computer program product described herein.

The current algorithm is for the practical application of the exposure-response model for selection of dose of C1-INH in individual patients in order to achieve optimal treatment of hereditary angioedema and/or optimal prevention of angioedema attacks.

The algorithm takes into account the number of HAE attacks in the past in treatment naïve patients or patients on standard fixed dose treatment along with the patients C1-INH functional activity. Based on this information; a patient's individual characteristic parameters are calculated using the pharmacokinetic and exposure-response models (Tozer and Rowland, Essentials of Pharmacokinetics and Pharmacodynamics, 2^{nd} edition, Wolters Kluwer 2016). The individual characteristic parameters are further used to predict the minimum dose that would ensure appropriate trough level C1-INH functional activity that would lead to the target optimal number of HAE attacks in a given period of time as shown in FIG. 2 and FIG. 4.

Presently, we provide an individualized dosing strategy. Further, we provide a comparison of the individualized dosing method vs. the currently used simple weight based dosing.

The dosing strategy provided herein relies on PK (C1-INH plasma levels) and PD (number of HAEA events) parameters obtained from individual patients. Herein, PK-PD is interchangeably called exposure-response (ER). These data are used to predict a dose resulting in an optimal treatment outcome. The provided method for determining a dosing scheme is advantageous compared to the standard-of-care (SOC) dosing.

### DESCRIPTION OF THE DRAWING

FIG. 1: Relationship between trough C1-inhibitor functional activity and relative risk. Example of applying the invention to an individual HAE patient with a baseline C1-INH activity of 25%. In order to achieve a, e.g., minimum 50% reduction in the relative risk of an HAE attack, this patient requires a dose that brings the C1-INH functional activity level above about 33% (C_{trough}). If, e.g., an 80% reduction in the relative risk of an HAE attack is desired, the dosing scheme would have to be adjusted to a C1-INH functional activity level of above about 46% (C_{trough}).

FIG. 2: SOC, TDM and TRUE Strategy

FIG. 3: Demonstration TDM Code for CSL830: For demonstration purposes, subject number 23 from the master simulation data is used. This 36 year old subject weighs 57.7 kg, and has a baseline C1-INH of 17.2. They had 10 attacks in the last 6 months on 60 IU/kg and 3 PK samples are 60.5, 63.2 and 65.9. The goal is to find the smallest dose giving a predicted count ≤6 for the second six months. All processing is done with NONMEM and SAS.

FIG. 4: Dose Selection Algorithm

FIG. 5: Scatterplot of Weight, Age, and Baseline C1-INH

FIG. 6: Distribution of Simulated HAE Counts for First 6 Months

FIG. 7: Simulated PK Responses for first 6 Months

FIG. 8: Percent Risk Reduction for Subjects not Controlled by 100 IU/kg

FIG. 9: Observed C1-INH Functional Activity versus Time After Dose

FIG. 10: Observed Baseline C1-INH Functional Activity by Subject Population

FIG. 11: Diagnostic Plots from Base Model

FIG. 12: Parameter ETA vs. Covariate plots (Base Model)

FIG. 13: Diagnostic Plots from Final Model

FIG. 14: Absolute Individual Weighted Residuals versus Individual Prediction

FIG. 15: Parameter ETA vs. Covariate plots (Final Model)

FIG. 16: Prediction-corrected Visual Predictive Check for the Final Population PK Model, Stratified by HAE Subjects and Healthy Volunteers; Open Circle: Observed Concentrations; Solid Line: Median of Observed Concentrations; Dashed Lines: 5th and 95th percentile of observed concentrations. Green Shaded Region: 95% Prediction Interval for Median of Predicted Concentrations; Blue Shaded Regions: 95% Prediction Intervals for the 5th and 95th percentiles of Predicted Concentrations

FIG. 17: Parameter ETA vs. Study (Final Model)

FIG. 18: Simulated Steady-State C1-INH Functional Activity After 40 IU/kg and 60 IU/kg Twice Weekly Dosing

FIG. 19: Observed C1-INH Antigen Concentrations versus Time After Dose

FIG. 20: Observed C1-INH Antigen Concentrations versus C1-INH Functional Activity by HAE Type

FIG. 21: Observed C4 Antigen Concentrations versus Time After Dose

FIG. 22: Observed C4 Antigen Concentrations versus C1-INH Functional Activity by HAE Type

FIG. 23: Observed C4 Antigen Concentrations versus C1-INH Antigen Concentrations by HAE Type

FIG. 24: ETA in CL vs. Covariate—Final Model (Run 012)

FIG. 25: ETA in V vs. Covariate—Final Model (Run 012)

FIG. 26: Representative Individual Observed and Predicted Concentration—Final Model (Run 012)

FIG. 27: Distributions of Interindividual Random Effects—Final Model (Run 012)

FIG. 28: Parameter ETA vs. Covariate plots—Base Model (008)

FIG. 29: Simulated Steady-state Trough C1-INH Functional Activity

FIG. 30: Individual Observed and Predicted Concentration—Final Model (Run 012)

FIG. 31: Observed C1-INH Functional Activity vs. Patients Receiving Rescue C1-INH within 1 Week of Study

FIG. 32: Parameter CL vs. Covariate plots—Final Model (012)

FIG. 33: Observed and Predicted Concentrations Stratified by Dose

### DETAILED DESCRIPTION

### Definitions

According to the present invention, the term “C1 esterase inhibitor” or “C1 inhibitor” (“C1-INH”) refers to the proteins or fragments thereof that function as serine protease inhibitors and inhibit proteases associated with the complement system, preferably proteases C1r and C1s as well as MASP-1 and MASP-2, with the kallikrein-kinin system, preferably plasma kallikrein and factor Xlla, and with the coagulation system, preferably factor Xla and factor XIIa. In addition, the C1-INH can serve as an anti-inflammatory molecule that reduces the selectin-mediated leukocyte adhesion to endothelial cells. C1-INH as used herein can be the native serine protease inhibitor or an active fragment thereof, or it can comprise a recombinant peptide, a synthetic peptide, peptide mimetic, or peptide fragment that provides similar functional properties, such as the inhibition of proteases C1r and C1s, and/or MASP-1 and MASP-2, and/or plasma kallikrein, and/or factor Xlla, and/or factor Xla. The term C1-INH shall also encompass all natural occurring alleles, splice variants and isoforms which have the same or similar functions as the C1-INH. For further disclosure regarding the structure and function of C1-INH, see U.S. Pat. Nos. 4,915,945, 5,939,389, 6,248,365, 7,053,176 and WO 2007/073186.

One “unit” (“U”) of C1-INH is equivalent to the C1-INH activity in 1 mL of fresh citrated plasma of healthy donors. The C1-INH may also be determined in “international units” (“IU”). These units are based on the current World Health Organization (WHO) standard for C1-INH concentrates (08/256) which was calibrated in an international collaborative study using normal local human plasma pools. In general, U and IU are equivalent.

The term “hereditary angioedema” (“HAE”) as used herein relates to angioedema caused by a low content and low inhibitory activity of C1-INH in the circulation (HAE type I) or by the presence of normal or elevated antigenic levels of C1-INH of low functional activity (HAE type II). The term “HAE” as used herein also encompasses HAE with normal C1-INH (also known as HAE type III) which has been described recently in two subcategories: (1) HAE due to mutation in the factor XII gene and, as a result, increased activity of factor XII leading to a high generation of bradykinin, and (2) HAE of unknown genetic cause. In patients suffering from hereditary angioedema, edema attacks can occur in various intervals, including a daily, weekly, monthly or even yearly basis. Furthermore, there are affected patients wherein no edema occurs.

The term “angioedema” (“edema”) as used herein relates to swelling of tissue, for example swelling of skin or mucosa. The swelling can occur, for example, in the face, at hands or feet or on the genitals. Furthermore, swelling can occur in the gastro-intestinal tract or in the respiratory tract. Other organs can also be affected. Swelling persists usually between one and three days. However, remission can already occur after hours or not until weeks.

The term “acute treatment” or “treatment” as used herein relates to the treatment of a patient displaying acute symptoms. Acute treatment can occur from the appearance of the symptom until the full remission of the symptom. An acute treatment can occur once or several times until the desired therapeutic effect is achieved.

The term “prophylactic treatment” or “prophylaxis” or “prevention” as used herein relates to the treatment of a patient in order to prevent the occurrence of symptoms. Prophylactic treatment can occur at regular intervals of days, weeks or months. Prophylactic treatment can also occasionally occur.

The term “trough level” or “trough concentration” as used herein is the lowest level (concentration) at which a medication is present in the body during treatment. Generally, the trough level is measured in the blood serum. However, local concentration within tissues may also be relevant. A trough level is contrasted with a “peak level”, which is the highest level of the medicine in the body, and the “average level”, which is the mean level over time.

The term “about” as used herein means within an acceptable error range for a particular value which partially depends on the limitations of the measurement system.

The term “C1-INH functional activity” or “C1-INH activity” as used herein refers to C1-INH functional activity as determined in a blood sample by, e.g., a commercially available functional chromogenic assay (e.g., Berichrom C1-Inhibitor (Siemens Healthcare Diagnostics)). 100% C1-INH functional activity is calculated as a percentage of mean normal activity (i.e. functional activity in samples from healthy volunteers).

### Method for Determining a C1-INH Dosing Scheme and Method for Adjusting a C1-INH Dosing Scheme

The present invention relates to a method for determining the optimal C1-INH dosing scheme for prophylaxis and/or treatment of an individual patient suffering from hereditary angioedema. In one embodiment, the provided method is for determining a dosing scheme for C1-INH for the treatment of hereditary angioedema. In a further embodiment, the provided method is for determining a dosing scheme for C1-INH for the prevention of hereditary angioedema attacks. By implementing this method, a dosing scheme is obtained that is optimized for the individual patient.

The provided method comprises the following steps:
(i) determining baseline C1-INH functional activity (Cr) in a sample obtained from the patient before C1-INH treatment,
(ii) predefining the desired relative risk reduction h(t),
(iii) determining the corresponding target C1-INH functional activity (Cp) based on a model, preferably a model based on formula

$$Cp = \frac{{e}^{3.4} \times ( log ( relative h ( t ) ) + \frac{- 10.5 \times Cr}{{e}^{3.4} + Cr} )}{- 10.5 - log ( relative h ( t ) ) - \frac{- 10.5 \times Cr}{{e}^{3.4} + Cr}}$$

wherein Cr is the baseline value determined in step (i) and relative h(t) is the desired relative risk reduction predefined in step (ii), and
(iv) determining the C1-INH dosing scheme required to maintain the patient's trough level C1-INH functional activity above the target C1-INH functional activity.

The baseline C1-INH functional activity in a sample obtained from a patient in step (i) can be measured by any standard means well-known in the art. In one embodiment, the baseline C1-INH functional activity is measured by a chromogenic assay. The sample obtained from a patient may be any sample, such as a tissue sample or a body fluid sample. In a preferred embodiment, the sample is a blood sample.

The relative reduction in the risk or an absolute number of occurrence of an angioedema attack in step (ii) may be selected in order to result in an optimal reduction of attacks. A patient experiencing a high frequency of attacks requires a higher relative reduction in the risk of occurrence of an angioedema attack than a patient experiencing angioedema attacks at a lower frequency in order to result in the same absolute treatment outcome. For example, a patient suffering from 20 attacks per year without treatment would suffer from 5 attacks per year upon risk reduction by 75%. A patient suffering from 10 attacks per year without treatment would suffer from 5 attacks per year upon risk reduction by already 50%.

In one embodiment, the desired relative reduction in the risk of occurrence of an angioedema attack for an individual patient is selected based on the frequency of attacks occurring in said patient. In a further embodiment, the desired relative reduction in the risk of occurrence of an angioedema attack for an individual patient is selected based on the severity of attacks occurring in said patient. In another embodiment, the desired relative reduction in the risk of occurrence of an angioedema attack for an individual patient is selected based on the frequency and/or based on the severity of attacks occurring in said patient.

The desired relative risk reduction may be individually selected in order to result in an outcome of any desired attack rate per year. In one embodiment, the desired relative risk reduction is selected in order to result in less than 10 attacks per year. In a further embodiment, the desired relative risk reduction is selected in order to result in less than 5 attacks per year. In another embodiment, the desired relative risk reduction is selected in order to result in less than 3 attacks per year. In a preferred embodiment, the desired relative risk reduction is selected in order to result in equal or less than 1 attack per year.

In a further embodiment, the desired relative risk reduction is selected in order to result in equal or less than 2 attacks per month. In another embodiment, the desired relative risk reduction is selected in order to result in equal or less than 1 attack per month.

The corresponding target C1-INH functional activity (Cp) required in the patient in order to achieve the desired risk reduction is determined in step (iii) based on a model.

In a preferred embodiment, the model allows determining Cp based on Cr and relative h(t), wherein Cr is the baseline value determined in step (i) and relative h(t) is the desired relative risk reduction predefined in step (ii).

In a more preferred embodiment, Cp is determined based on a model using the formula

$$Cp = \frac{{e}^{3.4} \times ( log ( relative h ( t ) ) + \frac{- 10.5 \times Cr}{{e}^{3.4} + Cr} )}{- 10.5 - log ( relative h ( t ) ) - \frac{- 10.5 \times Cr}{{e}^{3.4} + Cr}}$$

wherein Cr is the baseline value determined in step (i) and relative h(t) is the desired relative risk reduction predefined in step (ii).

In one embodiment, the corresponding target C1-INH functional activity (Cp) may vary by +/−50% around the determined value. In a further embodiment, the corresponding target C1-INH functional activity (Cp) may vary by +/−25% around the determined value. In another embodiment, the corresponding target C1-INH functional activity (Cp) may vary by +/−10% around the determined value. In yet another embodiment, the corresponding target C1-INH functional activity (Cp) may vary by +/−5% around the determined value. In yet another embodiment, the corresponding target C1-INH functional activity (Cp) may vary by +/−3% around the determined value. In yet another embodiment, the corresponding target C1-INH functional activity (Cp) may vary by +/−1% around the determined value.

The dosing scheme required in order to maintain the target C1-INH functional activity above the corresponding target C1-INH functional activity determined in step (iii) is determined in step (iv). The determination of the dosing scheme may involve analysis of C1-INH levels in a sample obtained from the patient, wherein the patient received a standard dose of C1-INH or several standard doses of C1-INH prior to obtaining the sample and an adjustment of the dosing scheme based on the C1-INH levels determined in the sample. The determination of the dosing scheme may also involve analysis of C1-INH levels in several samples obtained from the patient, wherein the patient received a standard dose of C1-INH or several standard doses of C1-INH prior to obtaining the samples and an adjustment of the dosing scheme based on the C1-INH levels determined in the samples. The sample may be any sample obtained from the patient. In one embodiment, the sample is a blood sample.

A method for determining a dosing scheme allowing the adjustment of C1-INH functional activity in a patient to a predefined value is, e.g., described in Zuraw et al. (Allergy, 2015, DOI:10.1111/a11.12658). The dosing scheme for an individual patient can also be determined using the model described in Example 3.

The present invention also relates to a method for adjusting a preexisting C1-INH dosing scheme for prophylaxis and/or treatment of an individual patient suffering from hereditary angioedema in order to optimize the treatment response. Accordingly, by implementing this method, a preexisting dosing scheme is altered resulting in an optimized dosing scheme for an individual patient. In one embodiment, the provided method is for adjusting a dosing scheme for C1-INH for the treatment of hereditary angioedema. In a further embodiment, the provided method is for adjusting a dosing scheme for C1-INH for the prevention of hereditary angioedema attacks.

The provided method comprises the following steps:
(i) determining baseline C1-INH functional activity (Cr) in a sample obtained from the patient before C1-INH treatment,
(ii) determining trough C1-INH functional activity in a sample obtained from the patient during ongoing treatment with a standard dose of C1-INH,
(iii) determining the optimal relative risk reduction h(t) based on the patient's treatment response to the treatment of step (ii),
(iv) determining the corresponding target C1-INH functional activity (Cp) based on a model, preferably a model based on formula

$$Cp = \frac{{e}^{3.4} \times ( log ( relative h ( t ) ) + \frac{- 10.5 \times Cr}{{e}^{3.4} + Cr} )}{- 10.5 - log ( relative h ( t ) ) - \frac{- 10.5 \times Cr}{{e}^{3.4} + Cr}}$$

wherein Cr is the baseline value determined in step (i) and relative h(t) is the desired relative risk reduction determined in step (iii), and
(v) determining the C1-INH dosing scheme required to maintain the patient's trough level C1-INH functional activity above the target C1-INH functional activity based on the trough C1-INH functional activity determined in step (ii).

Step (i) of the method for adjusting a dosing scheme may be carried out as described above for the method for determining a dosing scheme, respectively.

The trough level C1-INH functional activity in a sample obtained from the patient can be measured by any standard means well-known in the art in step (ii). In one embodiment, the trough level C1-INH functional activity is measured by a chromogenic assay. The sample obtained from a patient may be any sample, such as a tissue sample or a body fluid sample. In a preferred embodiment, the sample is a blood sample. In one embodiment, the sample has been obtained after treatment of the patient with one standard dose of C1-INH. In another embodiment, the sample has been obtained after treatment of the patient with several standard doses of C1-INH. In yet another embodiment, the sample has been obtained after C1-INH steady-state levels are achieved in the patient. In one embodiment, the standard dose is 40 U/kg administered twice a week. In another embodiment, the standard dose is 60 U/kg administered twice a week. In yet another embodiment, the standard dose is the dose indicated in the label of a C1-INH preparation.

The optimal relative risk reduction required or an absolute number of occurrence of an angioedema attack is determined in step (iii) based on the individual patient's response to the treatment of step (ii). For example, upon insufficient treatment response to a standard starting dose of a C1-INH starting dose, a more desired outcome in terms of relative risk reduction is selected which results in an optimized preventive treatment.

In one embodiment, the desired relative reduction in the risk of occurrence of an angioedema attack for an individual patient is selected based on the frequency of attacks occurring in said patient. In a further embodiment, the desired relative reduction in the risk of occurrence of an angioedema attack for an individual patient is selected based on the severity of attacks occurring in said patient. In another embodiment, the desired relative reduction in the risk of occurrence of an angioedema attack for an individual patient is selected based on the frequency and/or based on the severity of attacks occurring in said patient.

The desired relative risk reduction may be individually selected in order to result in an outcome of any desired attack rate per year. In one embodiment, the desired relative risk reduction is selected in order to result in less than 10 attacks per year. In a further embodiment, the desired relative risk reduction is selected in order to result in less than 5 attacks per year. In another embodiment, the desired relative risk reduction is selected in order to result in less than 3 attacks per year. In a preferred embodiment, the desired relative risk reduction is selected in order to result in equal or less than 1 attack per year.

In a further embodiment, the desired relative risk reduction is selected in order to result in equal or less than 2 attacks per month. In another embodiment, the desired relative risk reduction is selected in order to result in equal or less than 1 attack per month.

After selection of the relative risk reduction, the target C1-INH functional activity (Cp) is determined in step (iv) as described above for the method for determining a dosing scheme, respectively. The variation of the Cp value as described above for the method for determining a dosing scheme also applies here.

Step (v) of the method for adjusting a dosing scheme may likewise be carried out as described above for the method for determining a dosing scheme, respectively.

The present invention also relates to the provision of a further method for adjusting a C1-INH dosing scheme for individual patients in order to achieve optimal treatment of hereditary angioedema and/or optimal prevention of angioedema attacks. The method for adjusting a dosing scheme for C1-INH for the treatment of hereditary angioedema and/or the prevention of hereditary angioedema attacks in an individual patient comprises the following steps:
(i) determining trough C1-INH functional activity in a sample obtained from the patient during ongoing treatment with a standard dose of C1-INH,
(ii) determining the optimal risk reduction h(t) based on the patient's treatment response to the treatment of step (i),
(iii) determining the corresponding target C1-INH functional activity (Cp) based on a model, preferably a model based on formula

h(t)=exp(0.08)*(age/42)\hat{ }1.05*exp((−10.5)*Cp/(exp(3.4)+Cp))
wherein h(t) is the risk reduction determined in step (ii), and
(iv) determining the C1-INH dosing scheme required to maintain the patient's trough level C1-INH functional activity above the target C1-INH functional activity (Cp) based on the trough C1-INH functional activity determined in step (i).

The trough level C1-INH functional activity in a sample obtained from the patient can be measured by any standard means well-known in the art in step (i). In one embodiment, the trough level C1-INH functional activity is measured by a chromogenic assay. The sample obtained from a patient may be any sample, such as a tissue sample or a body fluid sample. In a preferred embodiment, the sample is a blood sample. In one embodiment, the sample has been obtained after treatment of the patient with one standard dose of C1-INH. In another embodiment, the sample has been obtained after treatment of the patient with several standard doses of C1-INH. In yet another embodiment, the sample has been obtained after C1-INH steady-state levels are achieved in the patient. In one embodiment, the standard dose is 40 U/kg administered twice a week. In another embodiment, the standard dose is 60 U/kg administered twice a week. In yet another embodiment, the standard dose is the dose indicated in the label of a C1-INH preparation.

The optimal risk reduction required or an absolute number of occurrence of an angioedema attack is determined in step (ii) based on the individual patient's response to the treatment of step (i). For example, upon insufficient treatment response to a standard starting dose of a C1-INH starting dose, a more desired outcome in terms of risk reduction is selected which results in an optimized preventive treatment.

In one embodiment, the reduction in the risk of occurrence of an angioedema attack for an individual patient is selected based on the frequency of attacks occurring in said patient. In a further embodiment, the reduction in the risk of occurrence of an angioedema attack for an individual patient is selected based on the severity of attacks occurring in said patient. In another embodiment, the reduction in the risk of occurrence of an angioedema attack for an individual patient is selected based on the frequency and/or based on the severity of attacks occurring in said patient.

The risk reduction may be individually selected in order to result in an outcome of any desired attack rate per year. In one embodiment, the risk reduction is selected in order to result in less than 10 attacks per year. In a further embodiment, the risk reduction is selected in order to result in less than 5 attacks per year. In another embodiment, the risk reduction is selected in order to result in less than 3 attacks per year. In a preferred embodiment, the risk reduction is selected in order to result in equal or less than 1 attack per year.

In a further embodiment, the risk reduction is selected in order to result in equal or less than 2 attacks per month. In another embodiment, the risk reduction is selected in order to result in equal or less than 1 attack per month.

The target C1-INH functional activity (Cp) is determined in step (iii) based on a model.

In a preferred embodiment, the model allows determining Cp based on h(t), wherein h(t) is the risk reduction determined in step (ii).

In a more preferred embodiment, Cp is determined based on a model using the formula

h(t)=exp(0.08)*(age/42)\hat{ }1.05*exp((−10.5)*Cp/(exp(3.4)+Cp)),

wherein h(t) is the risk reduction determined in step (ii).

The variation of the Cp value as described above for the method for determining a dosing scheme also applies here.

Step (iv) of the method for adjusting a dosing scheme may likewise be carried out as described above for the method for determining a dosing scheme, respectively.

In yet another embodiment, the present invention relates to a method for determining a therapeutic C1-INH concentration (Cp) for the treatment of hereditary angioedema and/or the prevention of hereditary angioedema attacks in an individual patient, using an age-dependent risk-for-an-attack model.

The model may involve the following parameters:
(i) background risk (B0),
(ii) effect of patient age on background risk (Age on B0),
(iii) maximum C1-INH effect (Eₘₐₓ), and
(iv) half maximal effective concentration of C1-INH (EC₅₀).

In one embodiment, the model is based on formula

$$h = {e}^{BO} \times {( \frac{age}{42} )}^{Age on B 0} \times e ( ( E max ) \times \frac{Cp}{( {e}^{EC 50} + Cp )} )$$

wherein h is the risk for an attack and age is the individual patient's age.

In one embodiment,
(i) B0 is between −0.665 and 0.825, preferably B0 is 0.0802,
(ii) Age on B0 is between 0.552 and 1.55, preferably Age on B0 is 1.05,
(iii) Eₘₐₓ is between −11.2 and −9.84, preferably Eₘₐₓ is −10.5
   and/or
(iv) EC₅₀ is between 3.16 and 3.64, preferably EC₅₀ is 3.4.

In one embodiment, the risk of occurrence of an angioedema attack is selected to result in equal or less than one attack per month. In a further embodiment, the risk of occurrence of an angioedema attack is selected to result in equal or less than one attack per three months. In a further embodiment, the risk of occurrence of an angioedema attack is selected to result in equal or less than one attack per six months. In yet a further embodiment, the risk of occurrence of an angioedema attack is selected to result in equal or less than one attack per year.

Also provided is a method for determining a dosing scheme for C1-INH for the treatment of hereditary angioedema and/or the prevention of hereditary angioedema attacks in an individual patient comprising the following steps:
(i) determining Cp according to the method described herein; and
(ii) determining the C1-INH dosing scheme required to maintain the patient's trough level C1-INH functional activity above Cp.

In one embodiment, the C1-INH dosing scheme is determined by using a one-compartmental pharmacokinetics model with first order absorption and first order elimination. In one embodiment, the one-compartmental pharmacokinetics model is weight-dependent. A method for determining a dosing scheme allowing the adjustment of C1-INH functional activity in a patient to a predefined value is, e.g., described in Zuraw et al. (Allergy, 2015, DOI:10.1111/a11.12658). The dosing scheme for an individual patient can also be determined using the model described in Example 3.

### Medical Use and Methods of Treatment

Also herein provided are medical uses and methods of treatment. In one embodiment, C1-INH for use in the treatment of hereditary angioedema is provided, wherein the dosing scheme for C1-INH is determined for an individual patient by the method for determining a dosing scheme described herein. In a further embodiment, C1-INH for use in the prevention of hereditary angioedema attacks is provided, wherein the dosing scheme for C1-INH is determined for an individual patient by the method for determining a dosing scheme described herein. In another embodiment, C1-INH for use in the treatment of hereditary angioedema is provided, wherein the dosing scheme for C1-INH is adjusted for an individual patient by the method for adjusting a dosing scheme described herein. In yet another embodiment, C1-INH for use in the prevention of hereditary angioedema is provided, wherein the dosing scheme for C1-INH is adjusted for an individual patient by the method for adjusting a dosing scheme described herein. Also provided is a method of treating hereditary angioedema in an individual patient, comprising administering C1-INH to the patient, wherein the dosing scheme is determined/adjusted by the method described herein. Further provided is a method of preventing hereditary angioedema attacks in an individual patient, comprising administering C1-INH to the patient, wherein the dosing scheme is determined/adjusted by the method described herein.

In a preferred embodiment, C1-INH is administered via subcutaneous administration. Upon subcutaneous administration, C1-INH functional activity time profiles exhibit a considerably lower peak-to-trough ratio and more consistent exposures after subcutaneous administration are achieved. Such lower peak-to-trough fluctuations are particularly desired for prophylactic treatment, as such relatively steady plasma levels ensure persistent protection from the occurrence of angioedema attacks in patients suffering from hereditary angioedema.

In a further embodiment, C1-INH is administered via intravenous administration. C1-INH may also be administered continuously by infusion or by bolus injection. C1-INH may also be administered by intra-arterial injection or intramuscular injection. In further embodiments, C1-INH may be administered to a patient by any pharmaceutically suitable means of administration. Various delivery systems are known and can be used to administer the composition by any convenient route. In one embodiment, the patient self-administers C1-INH.

In one embodiment, the invention relates to a kit comprising (i) a pharmaceutical composition comprising C1-INH, and (ii) instructions for carrying out the method for determining a dosing scheme described herein and/or instructions for using the computer program product described herein. In a further embodiment, the invention relates to a kit comprising (i) a pharmaceutical composition comprising C1-INH, and (ii) instructions for carrying out the method for adjusting a dosing scheme described herein and/or instructions for using the computer program product described herein. In one embodiment, the pharmaceutical composition comprising C1-INH is formulated for subcutaneous administration.

### Computer Program Product, Computer and Device

The present invention provides a computer program product stored on a computer usable medium, comprising: computer readable program means for causing a computer to carry out one of the methods described herein. Further provided is a computer comprising such a computer program product. Also provided is a device for determining a dosing scheme for C1-INH for the treatment of hereditary angioedema and/or the prevention of hereditary angioedema attacks in an individual patient comprising: (i) a unit for analyzing C1-INH activity in a sample obtained from the patient, and (ii) a computer comprising a computer program product stored on a computer usable medium as described herein. In one embodiment, the unit comprises means for carrying out a fully automated C1-INH assay. The C1-INH assay may be a chromogenic assay. The result of the C1-INH activity assay may be used by the computer for calculating the dosing scheme in order to result at a certain C1-INH activity. The sample may be a blood sample. In one embodiment, one sample is used for determining the dosing scheme. In a further embodiment, two or more samples are used for determining the dosing scheme. The samples may be measured simultaneously or subsequently.

In one embodiment, the present invention relates to a computer program product stored on a computer usable medium, comprising: computer readable program means for causing a computer to carry out the following steps:
(a) determining the corresponding target C1-INH functional activity (Cp) based on a model, preferably a model based on the formula

$$Cp = \frac{{e}^{3.4} \times ( log ( relative h ( t ) ) + \frac{- 10.5 \times Cr}{{e}^{3.4} + Cr} )}{- 10.5 - log ( relative h ( t ) ) - \frac{- 10.5 \times Cr}{{e}^{3.4} + Cr}}$$

for a predefined relative risk reduction (h(t)) in the risk of occurrence of an angioedema attack in a patient, wherein Cr is the C1-INH activity baseline value in the patient, and
(b) determining the C1-INH dosing scheme required to maintain the patient's trough C1-INH functional activity above the target C1-INH functional activity.

In another embodiment, the present invention relates to a computer program product stored on a computer usable medium, comprising: computer readable program means for causing a computer to carry out the following steps:
(a) determining the corresponding target C1-INH functional activity (Cp) based on a model, preferably a model based on the formula

h(t)=exp(0.08)*(age/42)\hat{ }1.05*exp((−10.5)*Cp/(exp(3.4)+Cp))
for a predefined risk reduction (h(t)) in the risk of occurrence of an angioedema attack in a patient,
(b) determining the C1-INH dosing scheme required to maintain the patient's trough C1-INH functional activity above the target C1-INH functional activity (Cp).

Further provided is a computer comprising a computer program product stored on a computer usable medium, comprising: computer readable program means for causing the computer to carry out steps (a) and (b) described above.

Also provided is a device for determining a dosing scheme for C1-INH for the treatment of hereditary angioedema and/or the prevention of hereditary angioedema attacks in an individual patient comprising: (i) a unit for analyzing C1-INH activity in a sample obtained from the patient, and (ii) a computer comprising a computer program product stored on a computer usable medium, comprising: computer readable program means for causing the computer to carry out steps (a) and (b) described above. In one embodiment, the unit comprises means for carrying out a fully automated C1-INH assay. The C1-INH assay may be a chromogenic assay. The result of the C1-INH activity assay may be used by the computer for calculating the dosing scheme in order to result at a certain C1-INH activity. The sample may be a blood sample. In one embodiment, one sample is used for determining the dosing scheme. In a further embodiment, two or more samples are used for determining the dosing scheme. The samples may be measured simultaneously or subsequently.

### C1 Esterase Inhibitor

In certain embodiments of the invention, the C1-INH is a plasma-derived or a recombinant C1-INH. In a preferred embodiment, C1-INH is plasma-derived. In further embodiments, C1-INH is identical to the naturally occurring human protein or a variant thereof. In other embodiments, the C1-INH is human C1-INH. C1-INH may be a recombinant analogue of human C1-INH protein.

C1-INH may be modified to improve its bioavailability and/or half-life, to improve its efficacy and/or to reduce its potential side effects. The modification can be introduced during recombinant synthesis or otherwise. Examples for such modifications are glycosylation, PEGylation and HESylation of the C1-INH or an albumin fusion of the described C1-INH. In some embodiments, C1-INH is a fusion construct between C1-INH and albumin, in particular human albumin. In some embodiments, the albumin is a recombinant protein. The C1-INH and albumin proteins may either be joined directly or via a linker polypeptide. For further disclosure regarding glycosylation and albumin fusion of proteins, see WO 01/79271 and WO 2016/070156.

### Preparation of C1-INH

The C1-INH can be produced according to methods known to the skilled person. For example, plasma-derived C1-INH can be prepared by collecting blood plasma from several donors. Donors of plasma should be healthy as defined in the art. Preferably, the plasma of several (1000 or more) healthy donors is pooled and optionally further processed. An exemplary process for preparing C1-INH for therapeutic purposes is disclosed in U.S. Pat. No. 4,915,945. Alternatively, in other embodiments, C1-INH can be collected and concentrated from natural tissue sources using techniques known in the art. Recombinant C1-INH can be prepared by known methods.

In certain embodiments, C1-INH is derived from human plasma. In further embodiments, C1-INH is prepared by recombinant expression.

A commercially available product comprising C1-INH is, e.g., plasma-derived Berinert® (CSL Behring). Berinert® is manufactured according to A. Feussner et al. (Transfusion 2014, 54: 2566-73) and is indicated for treatment of hereditary angioedema and congenital deficiencies. Alternative commercially available products comprising C1-INH are plasma-derived Cetor® (Sanquin), Cinryze® (Shire), and recombinant Ruconest®/Rhucin® (Pharming).

### EXAMPLES

### Example 1

To assess the relationship between C1-inhibitor functional activity and clinical response endpoints, a population-based pharmacokinetic-pharmacodynamic analysis was conducted using data from 90 patients who were randomized and treated (40 IU/kg vs Placebo or a 60 IU/kg vs Placebo treatment sequence; twice weekly, subcutaneous, self-administration). An interval censored repeated Time to Event (TTE) model was developed that allowed the ability to directly relate C1-INH functional activity at the time of attack to the HAE attack event. The final model consisted of two components: background (baseline) hazard and a drug effect in the form of a nonlinear maximum effect (Emax) function. Full model development included the addition of a random effect on the baseline hazard parameter (B0).

After development of the base model and addition of a random effect on B0, covariate testing was performed for the effect of age, weight, sex, baseline C1-inhibitor functional activity, baseline HAE attack count (attacks during run in period), and HAE type on the B0 parameter estimate. The final model only included the effect of age on background hazard B0.

The covariate analysis for a population of subjects with HAE from 12 to 72 years of age revealed that the baseline risk of HAE attack increased with age; younger subjects had a lower baseline risk compared with older subjects. The analysis also revealed that the effect of C1-INH in reducing the risk of HAE attack was not dependent on age. The key parameter estimates of the final model included an Emax (maximum fractional reduction in the risk of an HAE attack) of 0.99, corresponding to an infinite dose, and a half maximal effective concentration (EC50) of 29.9% for C1-inhibitor functional activity. This model demonstrated a strong exposure-response relationship, with increasing C1-inhibitor functional activity decreasing the absolute risk of experiencing an HAE attack.

The final population TTE model equation for absolute hazard of a breakthrough HAEA is as follows:

h(t)=exp(0.08)*(age/42)\hat{ }1.05*exp((−10.5)*Cp/(exp(3.4)+Cp)).

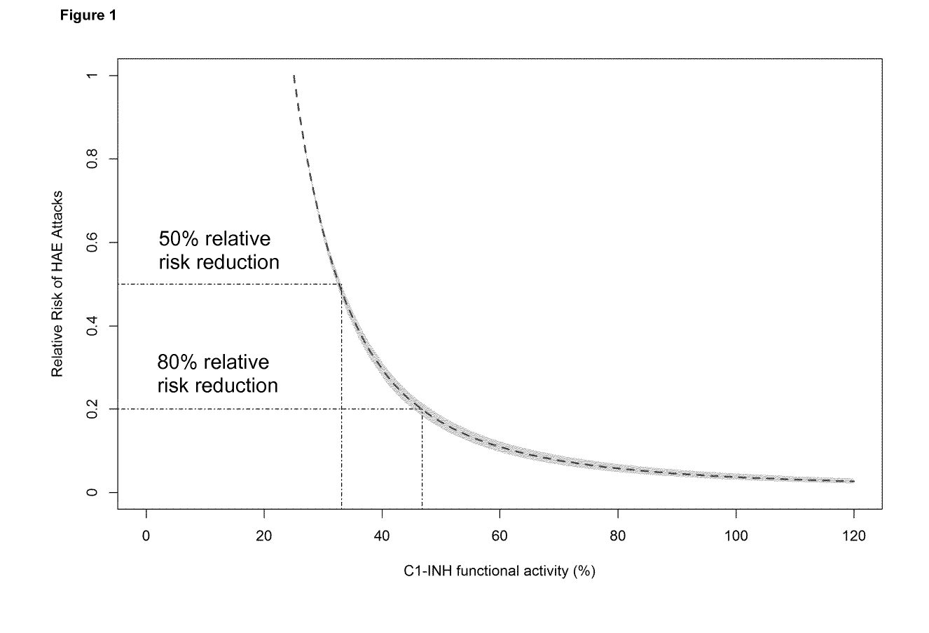

Based on the final model, reduction in the relative risk of experiencing an HAE attack compared to no prophylaxis treatment was calculated using the following equation across a wide range of C1-INH, ranging from 20% to 120%:

$$Relative h ( t ) = \frac{{e}^{( \frac{- 10.5 \times Cp}{{e}^{3.4} + Cp} )}}{{e}^{( \frac{- 10.5 \times Cr}{{e}^{3.4} + Cr} )}}$$

wherein Cp is C1-inhibitor functional activity, and Cr is the observed baseline reference C1-inhibitor functional activity before the beginning of treatment (In this example a value of 25% is used as reference) (FIG. 1).

### Example 2

CSL830 is a high concentration, volume-reduced formulation of plasma-derived C1-INH for routine prophylaxis against HAE attacks by the SC route of administration. It is available as a sterile, lyophilized powder in a single-use vial containing 1,500 International Units (IU) for reconstitution with 3 mL of diluent (water for injection). Subcutaneous (SC) injection relative to IV infusion represents a potentially safer, more easily and practically administered at-home prophylactic treatment option for HAE patients whose disease warrants long-term C1-INH therapy. C1-INH when administered SC twice weekly is expected to provide stable steady-state plasma levels and overall higher trough plasma levels relative to IV administration.

Current dosing practice (standard of care or SOC) for CSL830 is SC administration of 60 IU/kg twice weekly. After approximately 6 months of treatment the dose may be reduced to 40 IU/kg if the event count in the previous 6 months was ≤6.

Therapeutic drug monitoring (TDM) involves individualizing drug dosing based upon pharmacokinetic (PK) and/or pharmacodynamic (PD) responses (Evans W E, Schentag J J, Jusko W J., Applied Pharmacokinetics: Principles of Therapeutic Drug Monitoring. 3^{rd} Ed. Vancouver Wash., Applied Therapeutics, 1992). Both TDM and SOC dosing were evaluated using simulation of PK and PD based upon a pharmaco-statistical model that was developed previously. This extended PK-PD model will be referred to as the TRUE model in this application. The purpose of the simulation study is to compare the performance of the TDM based dosing with that based upon SOC dosing to provide patients the most optimal available care.

### Objectives

The objectives of these simulations/analyses are:
Develop a TDM strategy.
Compare the TDM and SOC dosing methods relative to the TRUE expected HAE count based on proportion of subjects attaining a predicted 6 month HAE count ≤6.
Compare the doses selected by the TDM, SOC, and TRUE strategies.
Explore risk reduction for subjects who are not predicted to have ≤6 HAE events in 6 months at the highest dose amount allowed in the TDM regimen.
Discuss alternative dosing strategies and assumptions implicit in this present work.

### Methods

### Overview of Strategies

For the first six months subjects all receive 60 IU/kg of CSL830 SC twice weekly. At the end of the first six months subjects report to the clinic with their HAE count for the previous six months (PD value). Everything up to this clinic visit is termed the history. At this clinic visit, a PK sample is obtained (the PK value is the C1-INH functional activity in the PK sample). PK samples are also obtained on the next two dosing days. The interval of collection for the three PK samples is termed the present. After a brief waiting period after the 3rd PK sample, termed the interim, the caregiver has the 3 PK concentrations based upon assay results. The interim duration is expected to be about one week beyond the time of the last PK sample. For this present work the interim will be ignored, in other words the PK samples have zero turnaround time.

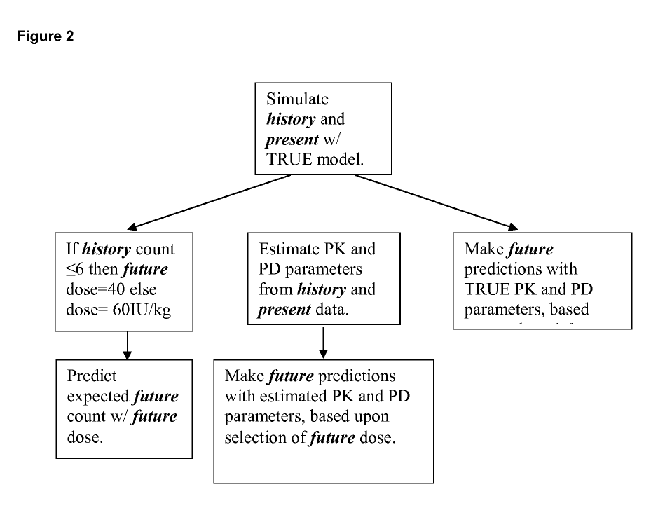

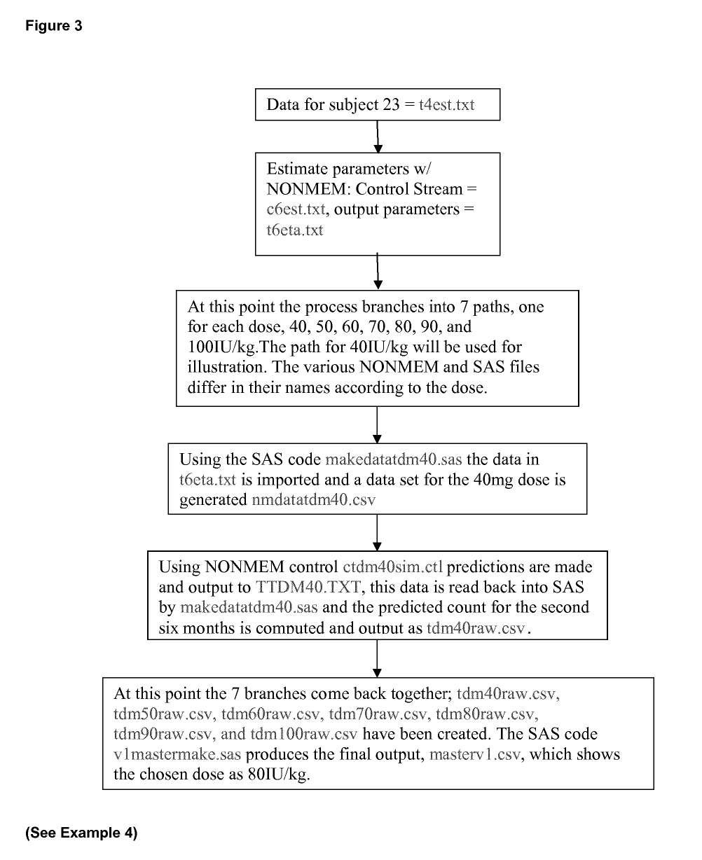

At this point a dose is chosen for the next six months. The next 6 months of follow-up and evaluate of HAE events is termed the future. Three methods of choosing the dose are evaluated. The first is the SOC method, which is based only upon the reported HAE count for the first six months; no model fitting is required for this approach. The second is the TDM approach, which requires empirical Bayes regression (model fitting) using the 3 PK concentration from the present and reported HAE counts from the history. That is, these data are fitted to produce a predicted PK profile and HAE count derived from the subject-specific parameter estimates. The third is the TRUE approach, which requires no model fitting. The TRUE approach uses the true subject-specific parameters from the simulation. For both the TDM and TRUE approaches, the expected number of HAE events for the future is predicted for all doses in permissible dose set {40, 50, 60, 70, 80, 90, and 100 IU/kg}. The smallest dose predicting a future expected number of HAE events <=6 is selected. If expected HAE events >6, the highest dose is retained (i.e., 100 IU/kg). The three strategies are displayed graphically in FIG. 2.

### The Models

Models describing the PK and PD (in terms of repeated measures time to HAE events) of CSL830 have been described previously (see Example 3). The PK model is parameterized in terms of baseline C1-INH, clearance (CL), volume of distribution (V), first order absorption rate (Ka) and bioavailability (F). The PK model has CL as a function of weight, and between subject variability on baseline, CL, V, Ka, and F (all log normal). Within subject (residual) variability is described with a proportional error model.

The time to event model hazard is composed of a baseline component, an age effect on baseline, and an Emax drug effect component driven by serum CSL830 concentration.

### Extending the PK-PD Model

For the time-to-event HAE model, the expected number of events over a time interval was taken to be the integral of the hazard function (i.e. the cumulative hazard) over that time interval. The HAE counts for the history were simulated using a truncated Poisson random variable. The mean was equal to the cumulative hazard from Week 2 to 6 months normalized to 6 months (24 weeks). This adjustment, was done because some subjects took 2-3 weeks to reach PK steady state.

### Simulation/Estimation/Prediction Specifics

Simulated data from 5000 virtual subjects are used for each simulation scenario. Dosing is assumed twice per week and the dosing times are assumed to be known accurately, such as through journal entry. True PK profiles are generated from the original PK model using bootstrapped values of weight and baseline. These PK profiles were input into the hazard function from the HAE time-to-event model, which was integrated to provide the expected number of HAE events for history. These computations were done using NONMEM 7.3.0 (ICON Development Solutions, Ellicot City, Md., USA). The expected number of HAE events for the history is exported and used as the mean for simulating Poisson random variable with an upper truncation point of 65. The motivation for truncation was to force the HAE response to be consistent with that observed in previous clinical studies. Without the truncation, some very large and clinically unrealistic HAE counts are generated, because the Poisson variable does not preclude risk of events explicitly during IV rescue after an HAE event. The C1-INH baseline, weight and age used in the PK and HAE models were simulated using a bootstrap procedure of data from previous clinical studies (2001 and 3001 studies). This simulation was done in the R language (http://www.r-project.org). SAS was used to construct and process data sets (SAS Institute Inc., SAS 9.1.3 Help and Documentation, Cary, N.C.: SAS Institute Inc., 2000-2004).

The TDM strategy requires subject specific estimation of the PK profile from PK samples collected during the present and simulated HAE counts from the history. The 3 observed PK samples are simulated for the present similar to the past, yet including residual variability. Information content of the PK samples with respect to estimating the subject-specific PK parameters depends upon the timing of the 3 PK samples. To account for variability due to sample timing in a realistic way, PK samples are assumed to be collected from 9 AM to 5 PM (distributed uniformly within the day). The day of the PK sample is selected with equal probability excluding Saturday and Sunday. Estimation of the subject-specific PK parameters was done in NONMEM using the Laplacian method with the MAXEVALS=0 and NOHABORT options. It should be noted that during the present and interim IV rescues do to HAE events were not incorporated to simplify the simulation strategy.

Finally, predictions of the expected counts, by dose for the second 6 months (future) were computed in NONMEM by integrating the hazard function. Dosing was assumed to be twice weekly. For the TDM approach, the subject-specific predicted PK profile was used along with the true HAE random effect for that subject when calculating the expected HAE event rate. Sample NONMEM and SAS code for one subject is presented in the Example 4.

### Dose Selection

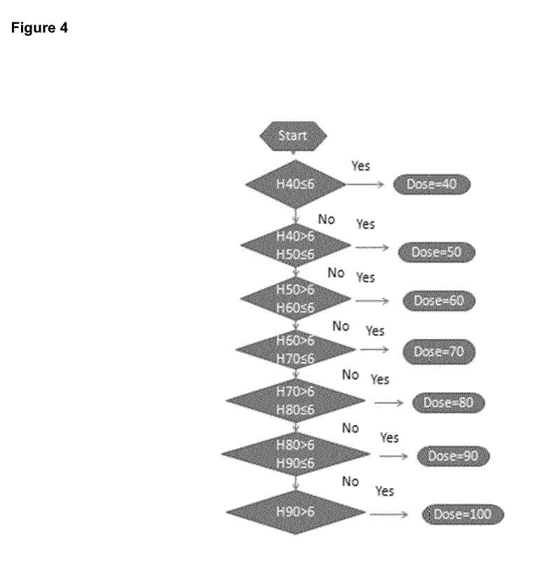

The dose selection for the SOC, TDM and TRUE strategy is presented in FIG. 2. Letting Hxy be the hazard function integrated over the second six months (predicted HAE count) for a dose of xy IU/kg, selection of the dose follows the flow diagram in FIG. 4. This algorithm is for the TDM and TRUE strategies, the only difference being that TDM uses estimated random effects and TRUE uses the (true) random effects used for simulation. In the case that Hxy is never ≤6, both the TDM and TRUE doses are truncated at 100 IU/kg, which is denoted as >100 for tabling purposes.

### Metrics for Reporting

The following metrics are of interest.
Proportion of subjects having a predicted HAE count for the second six months ≤6.
Distribution of selected doses by strategy.
Concordance of TRUE and TDM doses.
Risk reduction for subjects without adequate HAE event control (i.e., HAE count >6) at 100 IU/kg (>100).

The risk reduction calculation is presented in Equation.

$$RR ( \% ) = \frac{H ( history ) - H ( future )}{H ( history )} 100$$

where RR stands for risk reduction and H(·) is the cumulative hazard function (integrated hazard).

### Results

### PK and HAE Simulations

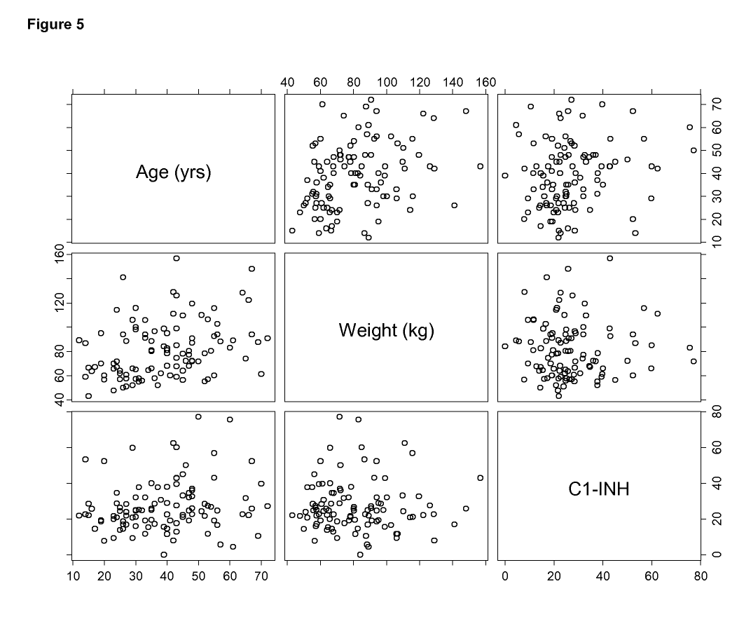

A total of 104 subjects from previous clinical studies (studies 2001 and 3001) had baseline C1-INH, weight and age. The relationships between the predictors are displayed in FIG. 5.

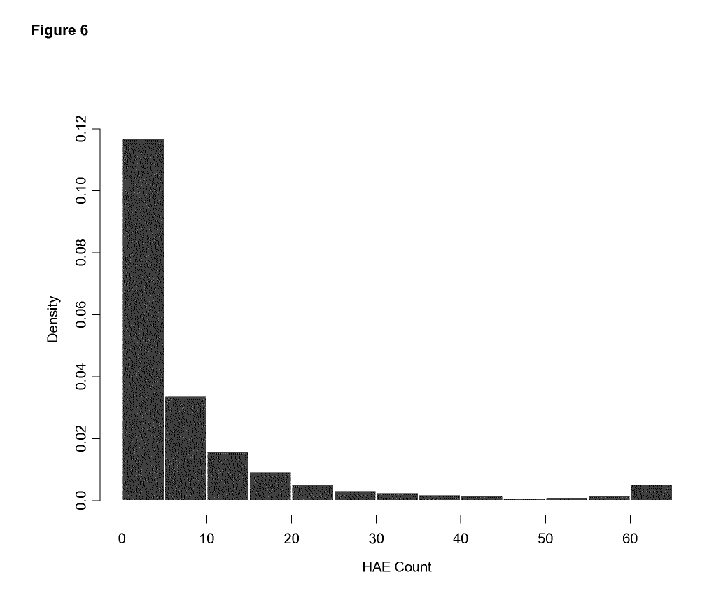

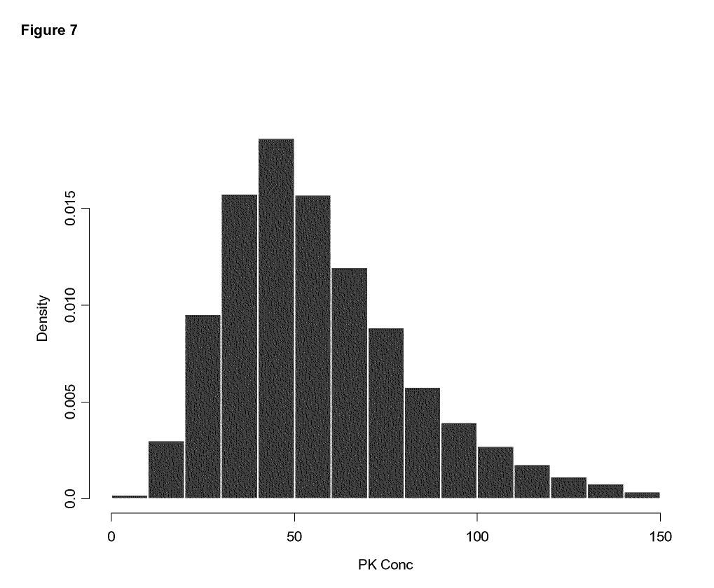

The simulated PK and PD values that are used for estimation are presented in Table 1, and FIGS. 6 and 7.

TABLE 1
Simulated PK and PD Values for First Six Months

| | | | | | | |
|---|---|---|---|---|---|---|
| PK | 1.72 | 38.1 | 51.9 | 70.9 | 147.4 | 362 |
| PD | 0 | 1 | 4 | 10 | 65 | 65 |
| (count) | | | | | | |

### Comparisons of Dosing Strategies

The number of subjects (out of 5000) attaining predicted HAE counts ≤6 for the second 6 months (future) were 2556, 3815, and 3890 for the SOC, TDM, and TRUE strategies, respectively. The distribution of doses selected by the three strategies is presented in Table 2.

TABLE 2
Dose Distribution for Second Six Months by Strategy

| | Dose (IU/kg) | | | | | | | |
|---|---|---|---|---|---|---|---|---|
| | 40 | 50 | 60 | 70 | 80 | 90 | 100 | >101 |
| SOC | 3146 | | 1854 | | | | | |
| TDM | 2234 | 410 | 283 | 283 | 228 | 202 | 175 | 1185 |
| TRUE | 2414 | 356 | 307 | 254 | 206 | 197 | 156 | 1110 |

SOC = Standard of care.
>101 indicates expected HAE count was >6.

In terms of concordance of doses compared to the TRUE dose, there was agreement in 2464/5000 and 3359/5000 subjects for the SOC and TDM doses, respectively.

In terms of risk reduction there are several considerations. Generally positive values are desirable. It should be noted that if the first 6 months (history) has a low cumulative hazard then for the TDM a smaller dose may be selected for the second 6 months (future) to get the E HAE <=6. This can generate negative risk reduction values.

Given that the goal is to up titrate dosages for those that are expected to have >6 HAE in 6 months and also to down titrate subjects to lower doses if over protected (which could increase counts), looking at risk reduction for the such an absolute threshold might seem intuitive. The percent risk reduction for the SOC and TDM dosing strategies are presented in Table 3.

TABLE 3
Percent Risk Reduction by Dosing Strategy

| | | | | | | |
|---|---|---|---|---|---|---|
| SOC | −196 | −77.9 | −48.5 | −9.2 | −1.3 | 28.6 |
| TDM | −188 | −67.9 | −31.1 | 35.4 | 62.1 | 69.0 |

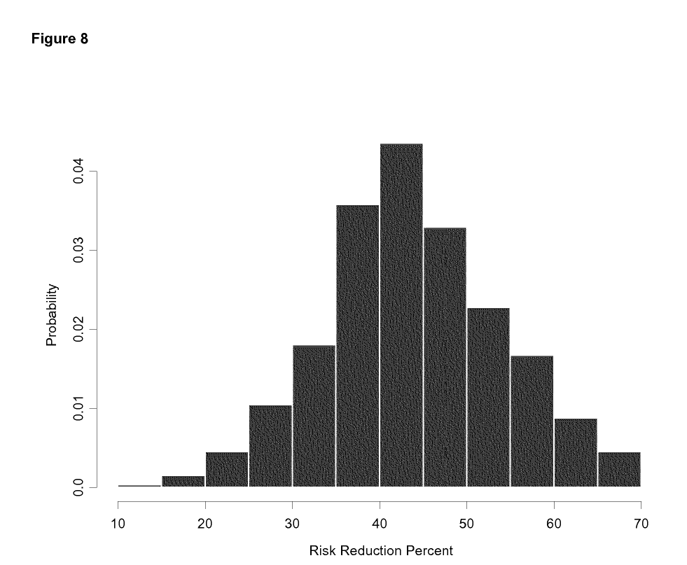

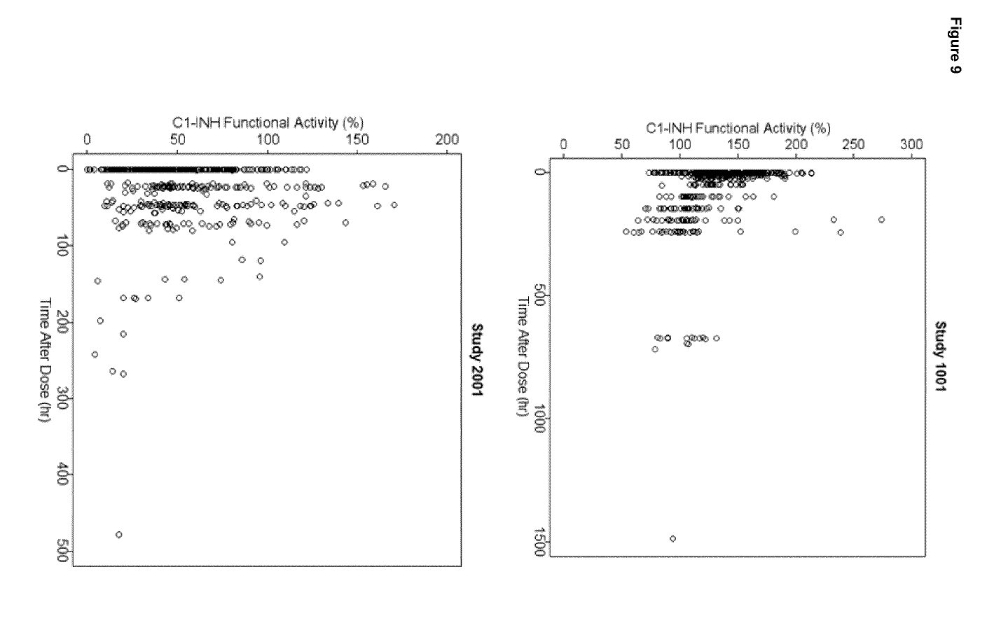

The subjects not controlled by 100 IU/kg (>100 population) for the TRUE or TDM strategies were evaluated further. Such subjects might still have a substantial decrease in disease severity. Risk reduction, as well as expected counts in the first, and second 6 months are stratified by TDM dose in Table 4. For those subjects not adequately titrated by 100 IU/kg, nearly 50% achieve a 43% risk reduction. The percent risk reduction for such patients is presented as a histogram in FIG. 8.

TABLE 4
Comparison of TDM for Controlled and Non-Controlled (>100) Subjects

| | Risk Reduction | | Expected Count 1^{st} 6 mos | | Expected Count 2nd 6 mos | |
|---|---|---|---|---|---|---|
| | | Not | | Not | | Not |
| | | Controlled | | Controlled | | Controlled |
| | Controlled | (>100) | Controlled | (>100) | Controlled | (>100) |
| Max | 69.0 | 68.8 | 17.3 | 68.6 | 6.00 | 49.8 |
| 99^{th} percentile | 51.7 | 67.6 | 11.5 | 67.8 | 5.98 | 41.2 |
| 75^{th} percentile | −8.91 | 50.5 | 5.21 | 38.6 | 5.46 | 20.7 |
| Median | −45.8 | 43.3 | 2.80 | 20.7 | 4.65 | 11.5 |
| 25^{th} percentile | −77.2 | 37.9 | 1.46 | 14.3 | 2.51 | 8.13 |
| Min | −188 | 12.9 | 0.172 | 7.61 | 0.288 | 6.00 |

### DISCUSSION

Based upon this work, TDM based dosing is promising compared to SOC dosing. The provided dosing model will provide an individually adjusted C1-INH dosing for patients resulting in an optimal treatment outcome.

### Example 3

Table of Contents

| 1 | LIST OF ABBREVIATIONS AND DEFINITIONS | | |
|---|---|---|---|
| 2 | SYNOPSIS | | |
| 3 | LIST OF TABLES | | |
| 4 | LIST OF FIGURES | | |
| 5 | LIST OF ATTACHMENTS | | |
| 6 | INTRODUCTION | | |
| 7 | OBJECTIVES | | |
| 8 | INVESTIGATIONAL PLAN | | |
| | 8.1 | STUDY POPULATION, DOSE REGIMENS, AND | |
| | PHARMACOKINETIC SAMPLING | | |
| | | 8.1.1 | Study 1001 |
| | | 8.1.2 | Study 2001 |
| | | 8.1.3 | Study 3001 |
| | 8.2 | BIOANALYTICAL METHODS | |
| | 8.3 | DATA RETRIEVAL | |
| | 8.4 | DATA REVIEW | |
| | 8.5 | ANALYSIS POPULATION | |
| | 8.6 | PHARMACOK1NETIC ANALYSES METHODS | |
| | 8.7 | POPULATION PHARMACOKINETIC ANALYSIS | |
| | | 8.7.1 | Base Model |
| | | 8.7.2 | Covariate Modeling |
| | 8.8 | MODEL EVALUATION AND DISCRIMINATION | |
| | 8.9 | FINAL MODEL EVALUATION | |
| | | 8.9.1 | Visual Predictive Check |
| | | 8.9.2 | Bootstrap Analysis |
| | 8.10 | SIMULATIONS | |
| | | 8.10.1 | Individual Predicted Pharmacokinetic Parameters |
| 9 | RESULTS | | |
| | 9.1 | DATASET ANALYZED | |
| | 9.2 | DEMOGRAPHICS AND COVARIATES | |
| | 9.3 | BASE MODEL DEVELOPMENT | |
| | 9.4 | CO VARIATE MODEL DEVELOPMENT | |
| | 9.5 | FINAL MODEL | |
| | 9.6 | FINAL MODEL EVALUATION | |
| | 9.7 | POSTHOC ANALYSIS | |
| | 9.8 | SIMULATIONS | |
| | 9.9 | EXPLORATORY ANALYSIS | |
| | | 9.9.1 | C1-INH Antigen |
| | | 9.9.2 | C4 Antigen |
| | | 9.9.3 | C1-INH Antigen vs. C4 Antigen |

| | |
|---|---|
| 10 | DISCUSSION |
| 11 | CONCLUSIONS |
| 12 | QUALITY CONTROL |
| 13 | REFERENCES |
| 14 | APPENDIX |
| 15 | ATTACHMENTS |

### 1 LIST OF ABBREVIATIONS AND DEFINITIONS

Note: Complete listing of data item abbreviations and descriptions as implemented in NONMEM datasets are provided in Table 7.

| Abbreviation | Definition |
|---|---|
| $COV | covariance command in NM-TRAN |
| $EST | estimation command in NM-TRAN |
| θ | fixed effect parameter (theta) |
| Θ | vector containing fixed effect parameters |
| ρ | correlation coefficient (rho) |
| Ω | variance-covariance matrix |
| η | random quantity at the individual level (eta) |
| ϵ | random quantity at the observation level (epsilon) |
| χ² | chi square |
| ω² | variance of inter-individual variability parameter η |
| σ² | variance of residual error quantity ϵ |
| AIC | Akaike Information Criterion |
| AUC | area under the serum/plasma drug functional |
| | activity-time curve |
| AUC_{0-τ} | Area under the serum/plasma drug functional |
| | activity-time curve from Pre-dose to the end |
| | of the dosing interval at steady state |
| BLQ | below the lower limit of quantification for a bioassay |
| BMI | body mass index |
| BSA | body surface area |
| CAT | categorical covariate |
| CI | confidence interval |
| CL/F | apparent oral clearance |
| Cₘₐₓ | maximum serum/plasma functional activity |
| C_{trough} | minimum (trough) serum/plasma functional |
| | activity t steady state |
| COV | continuous covariate |
| CRCL | creatinine clearance |
| CV | coefficient of variation |
| CWRES | conditional weighted residual |
| C_{τ} | concentration at the end of a dosing interval |
| d.f. | degrees of freedom |
| DV | dependent variable (also Y_{obs}) |
| e | base of the natural logarithm |
| EMA | European Medicines Agency |
| EVID | event identification NONMEM data item |
| F | model prediction of the dependent variable (also Y_{pred}) |
| FDA | US Food and Drug Administration |
| FOCEI | First-order Conditional Estimation method with |
| | Interaction |
| GAM | Generalized Additive Modeling |
| GoF | goodness-of-fit |
| HAEA | Hereditary Angioedema Attack |
| IIV | inter-individual variability |
| IMP | Monte Carlo Importance Sampling Expectation |
| | Maximization method |
| IPRED | individual prediction |
| ITS | Iterative Two Stage method |
| IV | intravenous |
| IWRES | individual weighted residuals |
| Ka | first-order rate of absorption |
| kg | kilogram |
| L | liter |
| LLQ | lower limit of quantification |
| MAP | Monte Carlo Importance Sampling Expectation |
| | Maximization Assisted by Mode a Posteriori method |
| mg | milligram |
| mL | milliliter |
| MSAP | Modeling and Simulation Analysis Plan |
| NA | not applicable |
| NONMEM | Non-Linear Mixed-Effects Modeling software |
| NQ | not quantified |
| OBS | observed serum/plasma concentration |
| OFV | objective function value |
| p | probability |
| P | pharmacokinetic parameter |
| PD | pharmacodynamics |
| PI | prediction interval |
| PK | pharmacokinetic(s) |
| PK/PD | pharmacokinetic/pharmacodynamic |
| Pop PK | population pharmacokinetics |
| PRED | population prediction |
| QC | quality control |
| QQ | quantile-quantile |
| RSE | relative standard error |
| SAEM | Stochastic Approximation Expectation Maximization |
| | method |
| SC | subcutaneous |
| SD | standard deviation |
| sh_{η} | shrinkage in the standard deviation of inter-individual |
| | variability parameter η |
| sh_{ϵ} | shrinkage in the standard deviation of individual |
| | weighted residuals |
| t_{1/2α} | drug elimination half-life in the initial disposition phase |
| t_{1/2β} | terminal drug elimination half-life |
| TV | typical value of a model parameter |
| V_{c} | volume of central compartment |
| Vₚ | volume of peripheral compartment |
| VPC | visual predictive checks |
| V_{c,ss} | volume of central compartment at steady-state |
| W | weighting factor for residual error structure |
| WBC | White Blood Cell |
| Y_{obs} | observed data (dependent variable) (also DV) |
| Y_{pred} | model prediction of the dependent variable (also F) |
| Yr | year |

### Conventions

In development, C1-esterase inhibitor human (subcutaneous [SC]) was also referred to as CSL830. In this document, the abbreviation CSL830 is used.

All studies summarized in this document are formally assigned the sponsor-assigned drug code, CSL830, followed by an underscore and a unique 4-digit number. For convenience to the reviewer, study numbers in this document are shortened to the unique 4-digit number. For example, Study CSL830_3001 is referred to as Study 3001.

### 2 SYNOPSIS

Title: Population Pharmacokinetic Analysis of CSL830 in Patients with Hereditary Angioedema

Phase of Development: I, II, III
Objectives:
The objectives of these analyses are:
To characterize the population PK of C1-INH functional activity in patients with HAE
To identify sources of variability in C1-INH functional activity PK
To perform the simulations based on the final population model to support dosing of CSL830
To perform exploratory evaluation of the correlation between C1-INH activity, C1-INH antigen
concentrations and C4 antigen concentrations
Methodology: Modeling The population C1-INH functional activity data in the subjects treated
with CSL830 (Studies 1001, 2001 and 3001) were analyzed by nonlinear mixed effects modeling
using the package NONMEM (v7.2). The base model comprised of a one-compartment model
with 2 separate baselines for patients and healthy volunteers. Absorption of CSL830 from the
subcutaneous depot site in to the central compartment was modeled as a 1^{st}-order process with
absorption rate constant (Ka, hour⁻¹).
Simulation One thousand individual profiles for the treatment-experienced population based on
the distribution of individual weights were simulated to derive relevant PK parameters.
Number of Subjects: 124
Results: The C1-INH functional activity following administration of CSL830 was adequately
described by a linear one-compartment model with first-order absorption, absorption and first-
order elimination, with inter-individual variability in all the parameters. The population mean
bioavailability of CSL830 was 0.427. Body weight effect on CL of C1-INH functional activity
was included in the final model with the weight exponents on CL estimated to be 0.738. The
population PK parameters CL, Vd, and Ka were estimated to be 0.830 IU/hr · %, 43.3 IU/%, and
0.0146 hr⁻¹, respectively.
The steady state simulations resulted in mean (95% CI) of steady-state Cₘₐₓ of 48.7 (26.9-96.2)
and 60.7 (31.8-128) and C_{trough} of 40.2 (22.2-77.9) and 48.0 (25.1-102) for 40 IU/kg and 60 IU/kg
doses respectively. The simulations derived median (95% CI) Tₘₐₓ was 58.7 (23-134) and half-life
was 36.9 (14.3-102) for both doses.
Conclusions:
C1-INH functional activity was well described by a one-compartment model with first order
absorption.
Body weight was a significant covariate that affected CL of CSL830.
Simulations at 40 IU/kg and 60 IU/kg twice weekly dose of CSL830 results in a mean C_{trough} of
40.2 and 48.0% C1-INH functional activity respectively.

### 3 LIST OF TABLES

Table 1 Summary of Studies Included in the Population PK Analysis
Table 2 Subject Characteristics and Demographics by Study
Table 3 Parameter Estimates of Base CSL830 Population PK Model
Table 4 Summary of Covariate Model Development
Table 5 Parameter Estimates of Final CSL830 Population PK Model
Table 6 Summary of Stead-State CSL830 Cₘₐₓ, Cₘᵢₙ and AUC_{0-τ} from the Simulated Population Stratified by Dose
Table 7 Data Item Abbreviations and Descriptions in the Dataset and NONMEM
Table 8: Summary of AUC Ratio (Multiple/Single Dose) for CSL830 Accumulation After Simulated 40 IU/kg or 60 IU/kg Twice per Week Dosing

### 4 LIST OF FIGURES

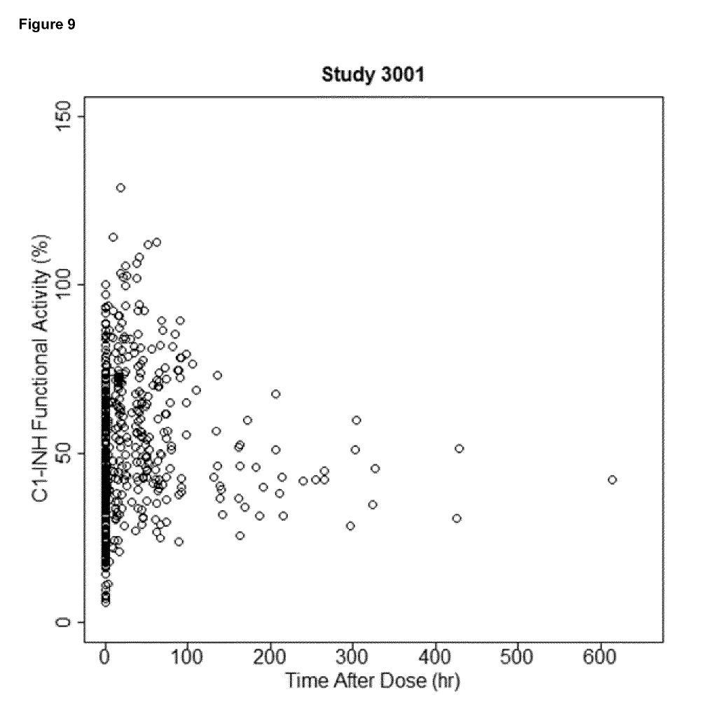

FIG. 9: Observed C1-INH Functional Activity versus Time After Dose

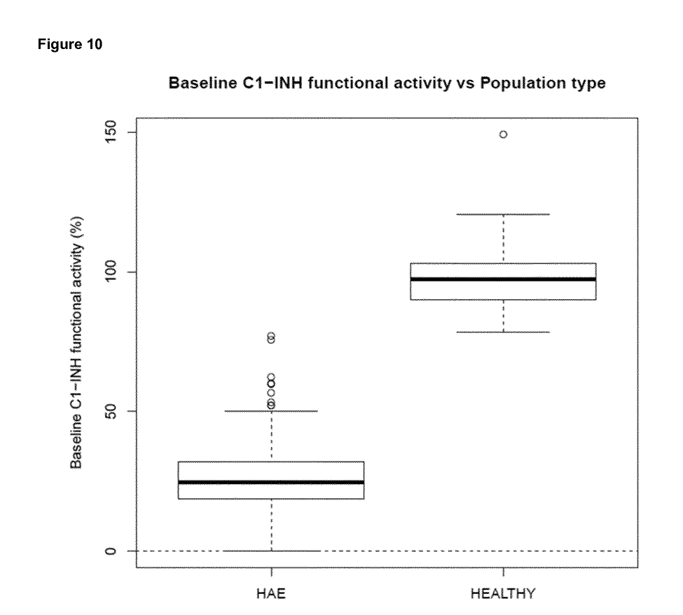

FIG. 10: Observed Baseline C1-INH Functional Activity by Subject Population

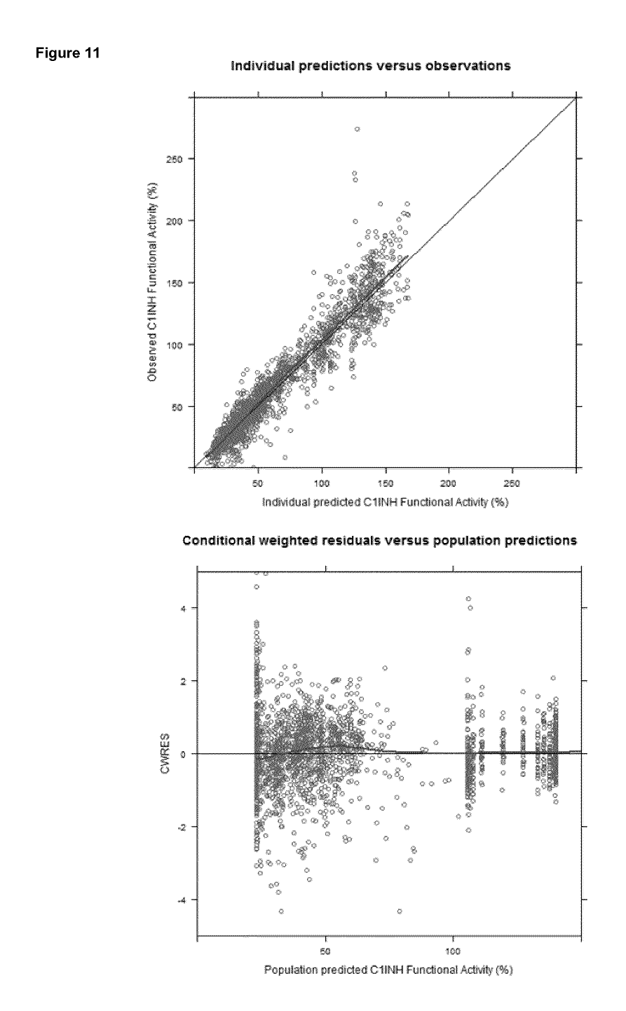

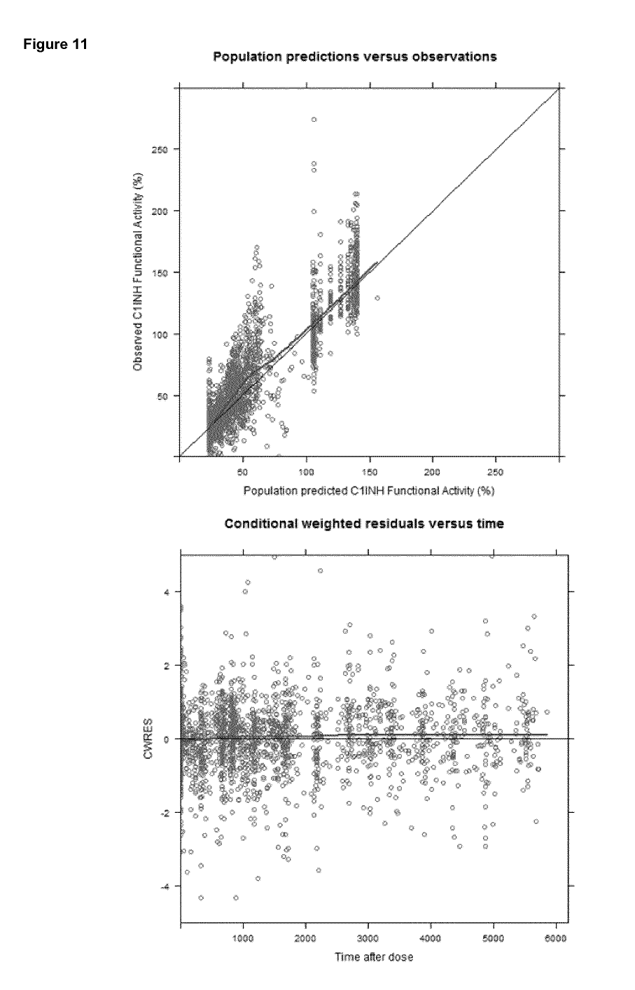

FIG. 11: Diagnostic Plots from Base Model

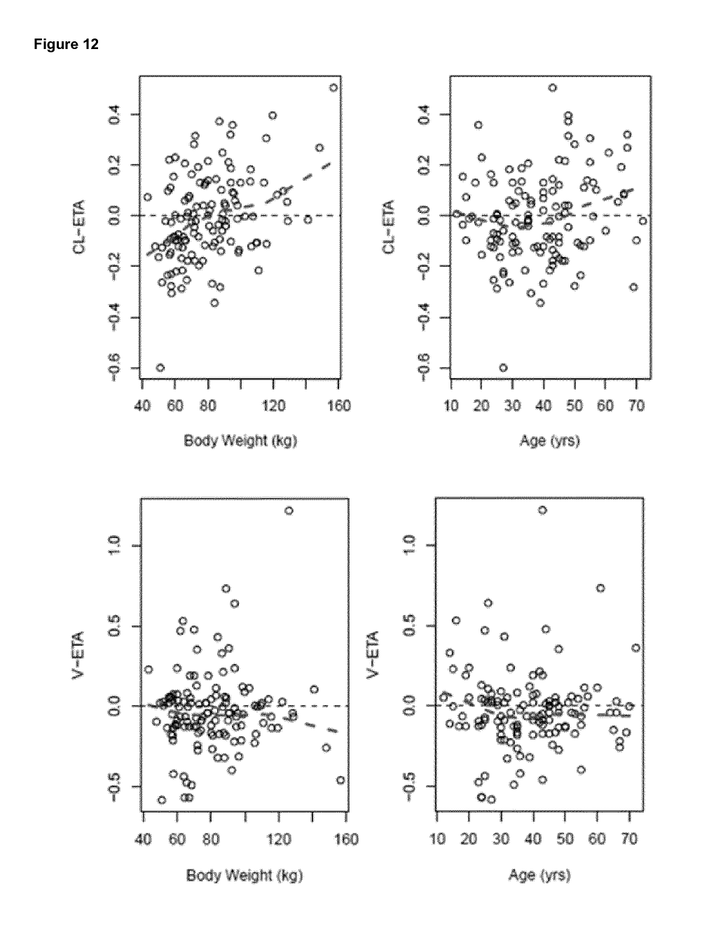

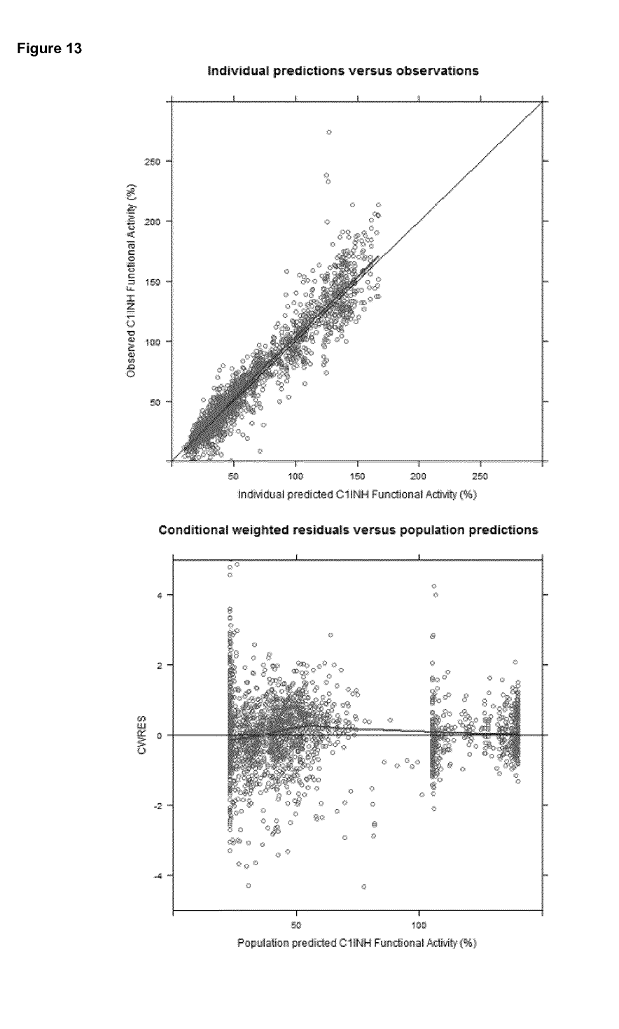

FIG. 12: Parameter ETA vs. Covariate plots (Base Model)

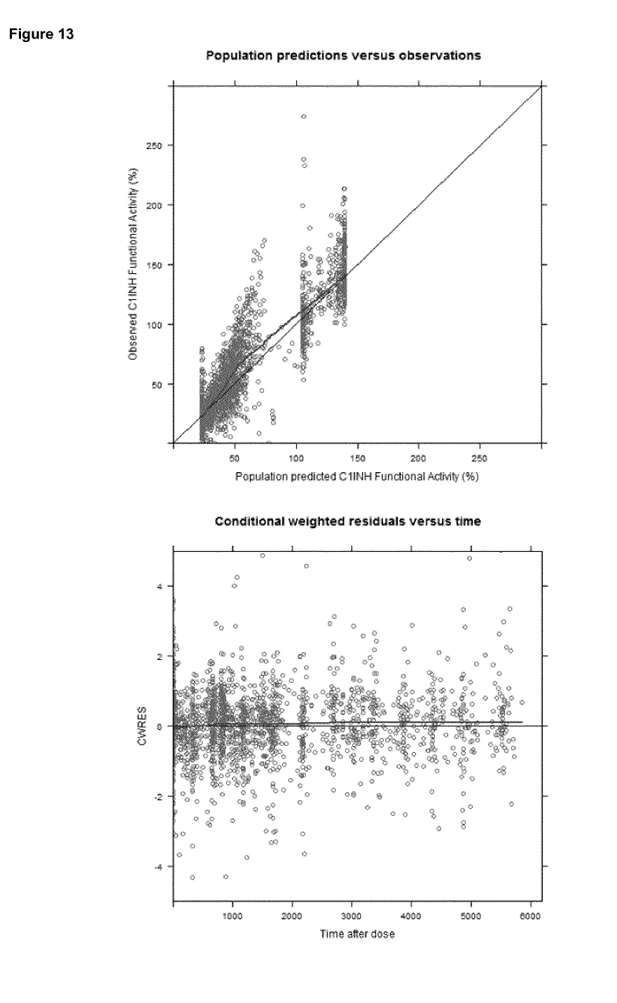

FIG. 13: Diagnostic Plots from Final Model

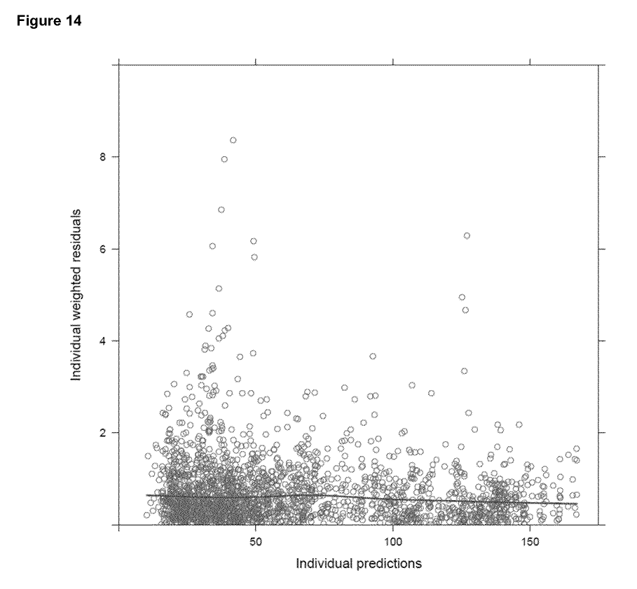

FIG. 14: Absolute Individual Weighted Residuals versus Individual Prediction

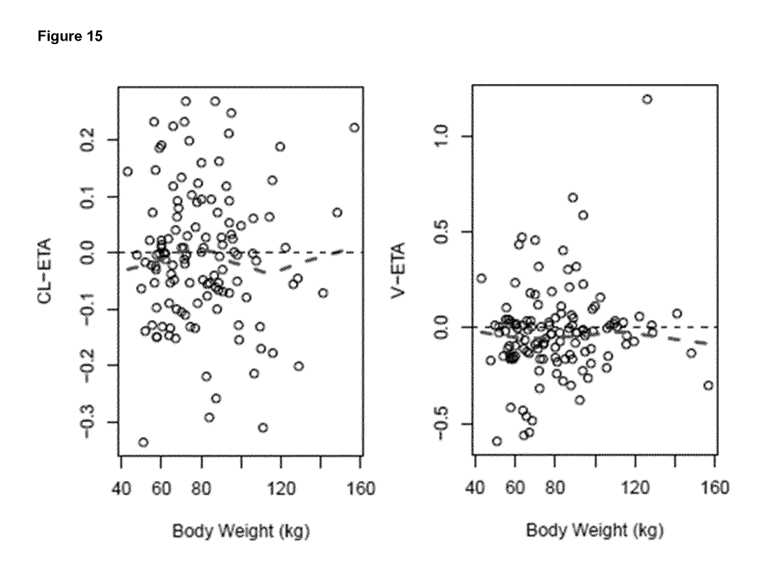

FIG. 15: Parameter ETA vs. Covariate plots (Final Model)

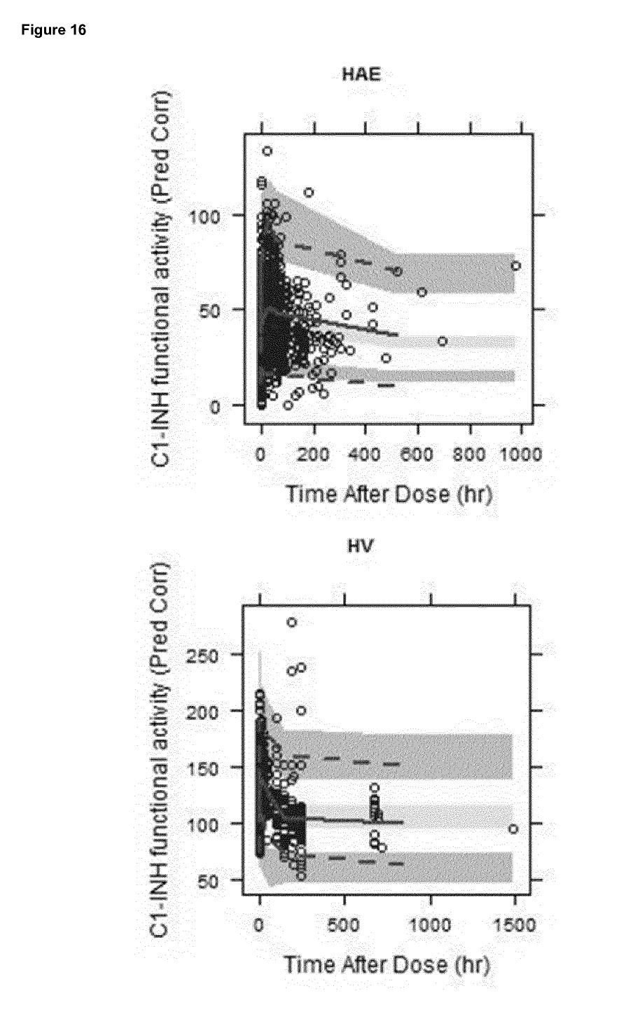

FIG. 16: Prediction-corrected Visual Predictive Check for the Final Population PK Model, Stratified by HAE Subjects and Healthy Volunteers; Open Circle: Observed Concentrations; Solid Line: Median of Observed Concentrations; Dashed Lines: 5th and 95th percentile of observed concentrations. Green Shaded Region: 95% Prediction Interval for Median of Predicted Concentrations; Blue Shaded Regions: 95% Prediction Intervals for the 5th and 95th percentiles of Predicted Concentrations

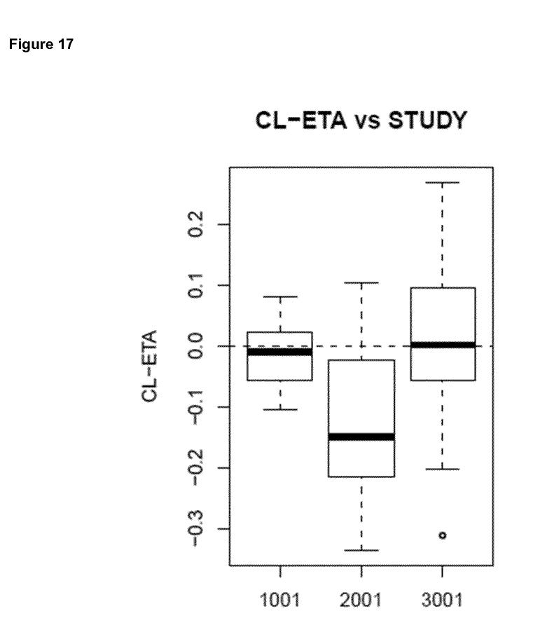

FIG. 17: Parameter ETA vs. Study (Final Model)

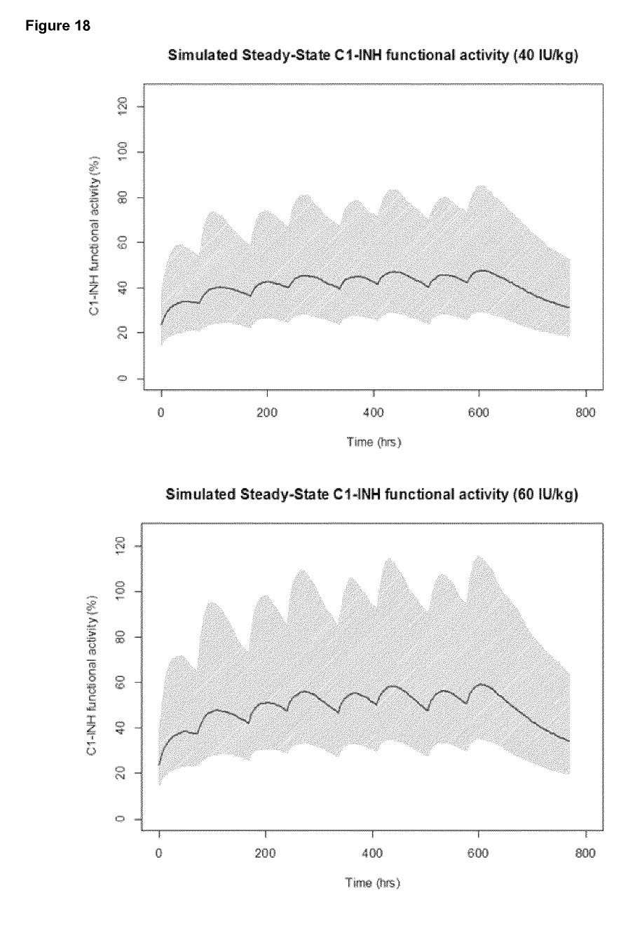

FIG. 18: Simulated Steady-State C1-INH Functional Activity After 40 IU/kg and 60 IU/kg Twice Weekly Dosing

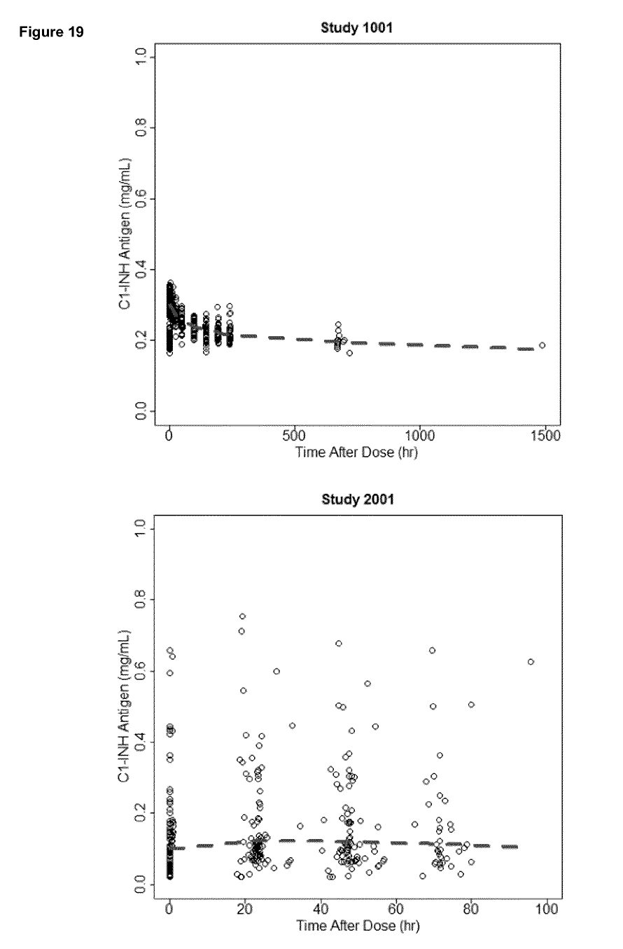

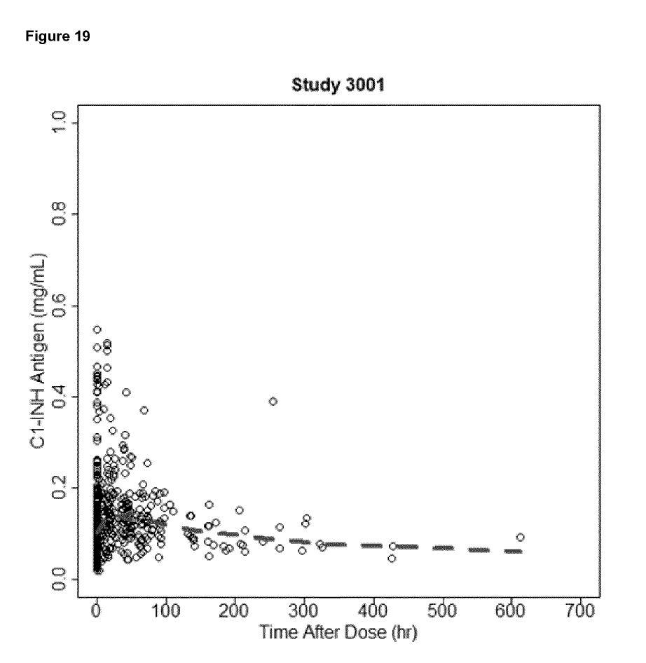

FIG. 19: Observed C1-INH Antigen Concentrations versus Time After Dose

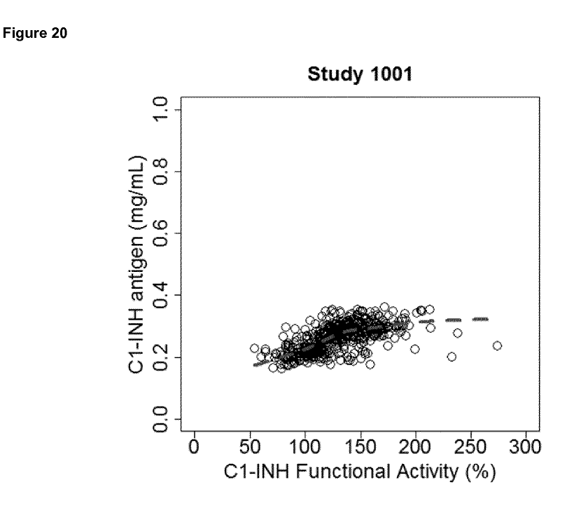

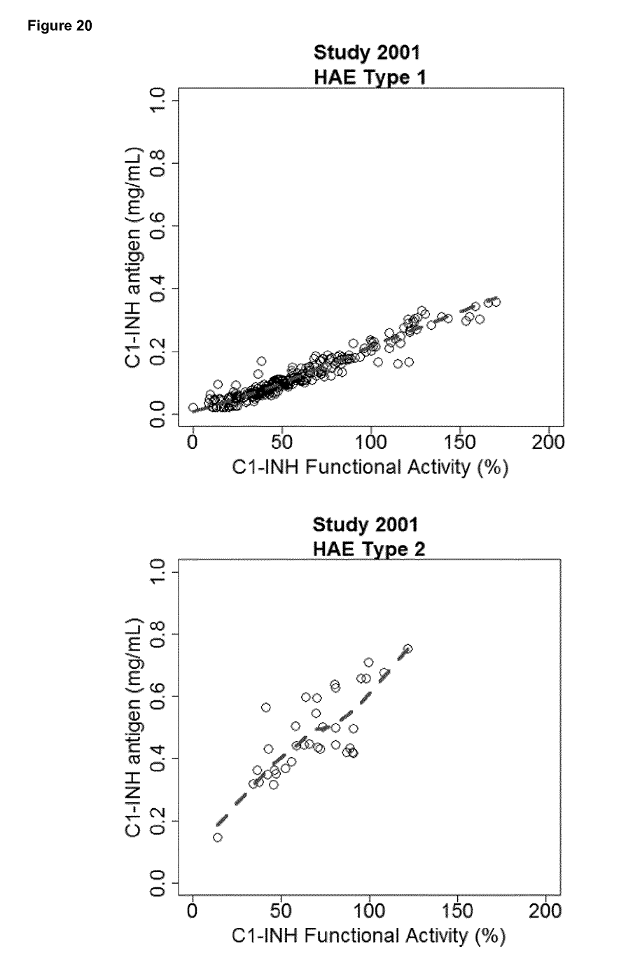

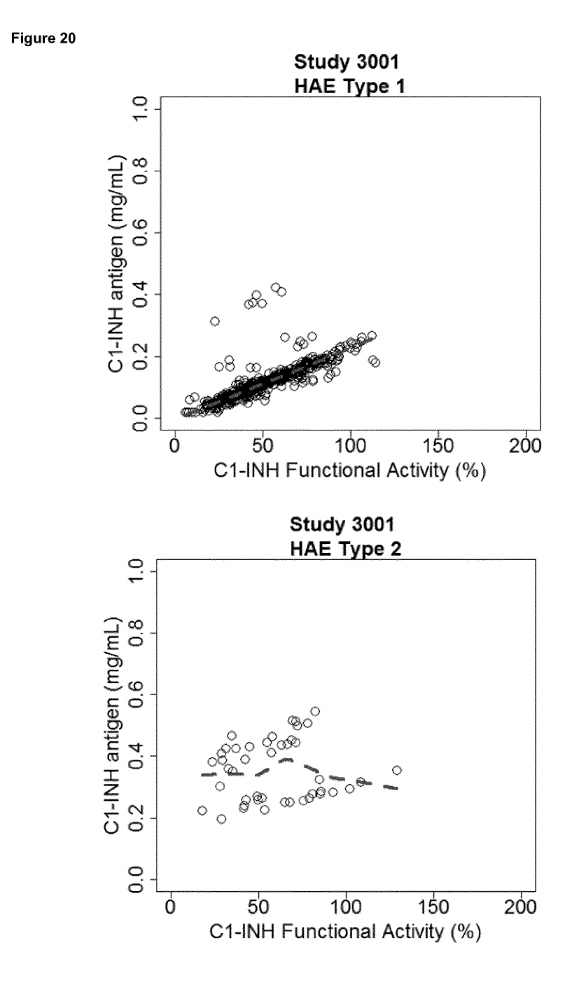

FIG. 20: Observed C1-INH Antigen Concentrations versus C1-INH Functional Activity by HAE Type

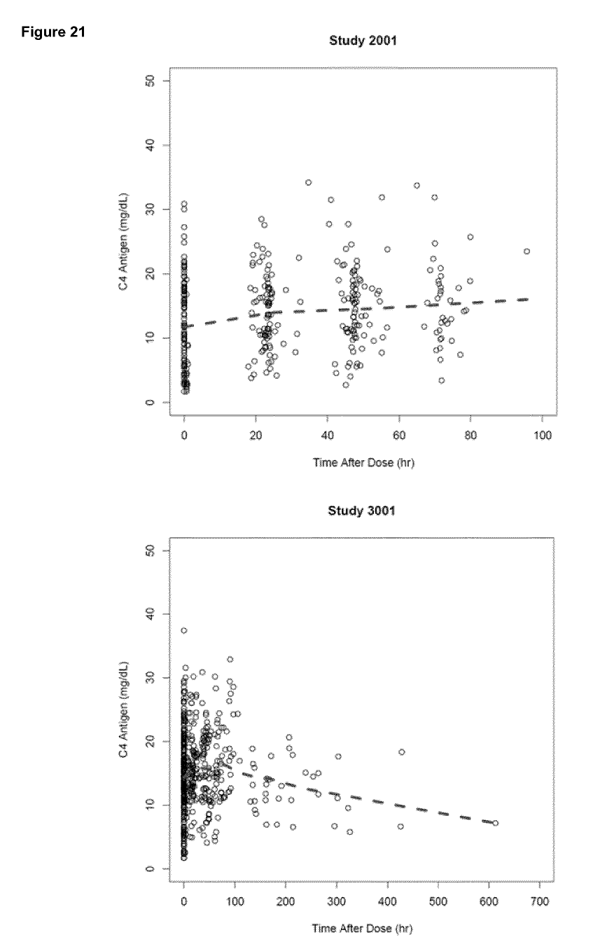

FIG. 21: Observed C4 Antigen Concentrations versus Time After Dose

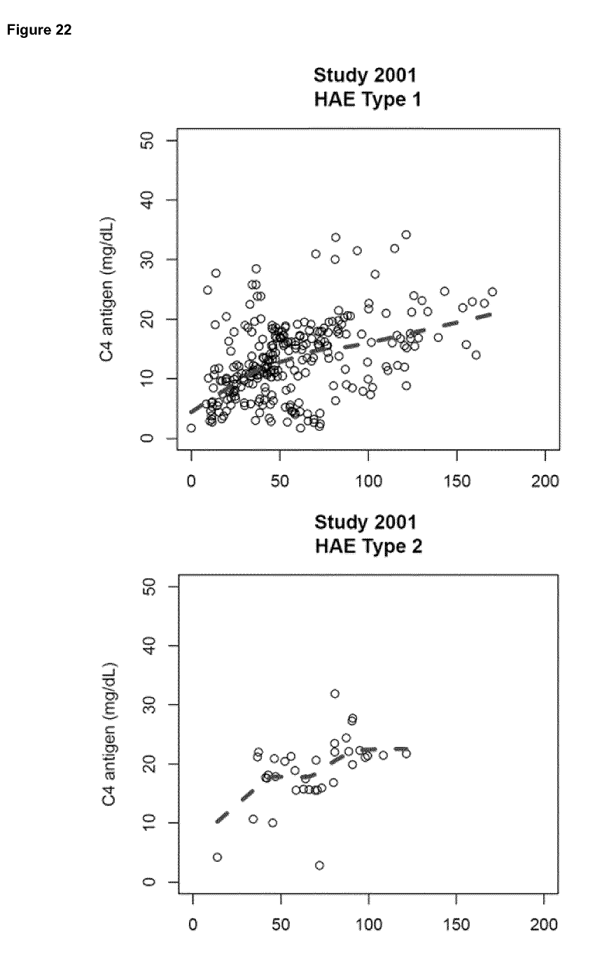

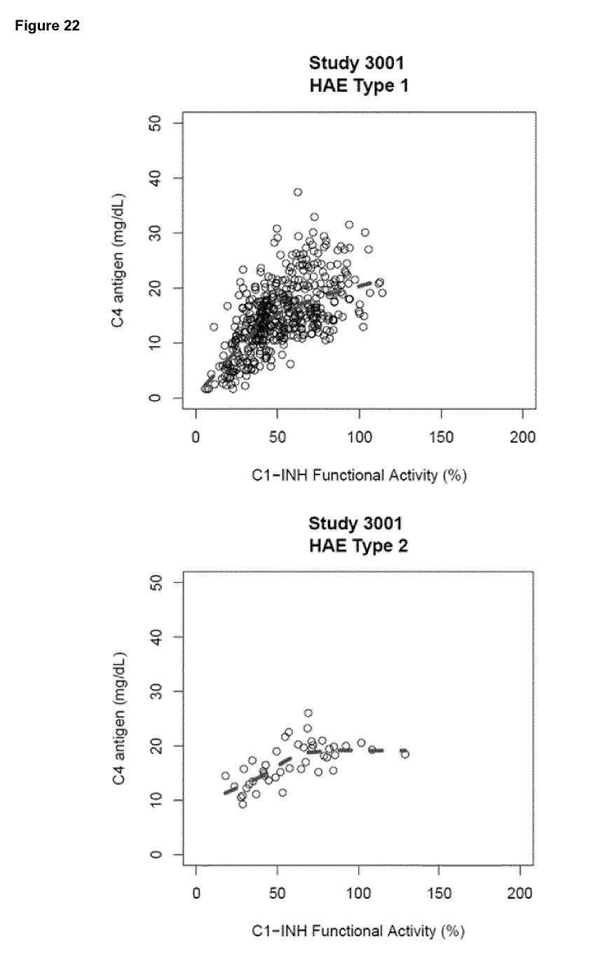

FIG. 22: Observed C4 Antigen Concentrations versus C1-INH Functional Activity by HAE Type

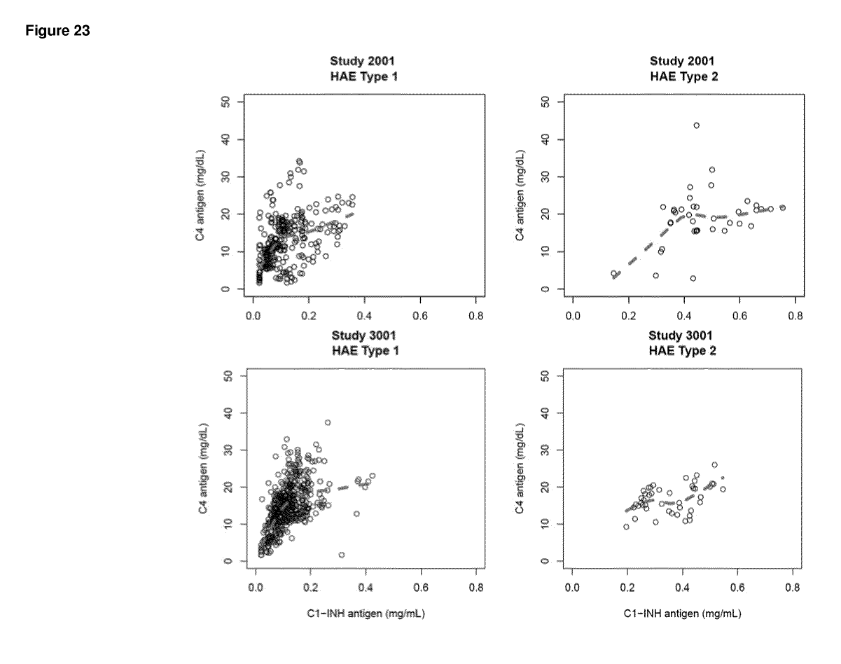

FIG. 23: Observed C4 Antigen Concentrations versus C1-INH Antigen Concentrations by HAE Type

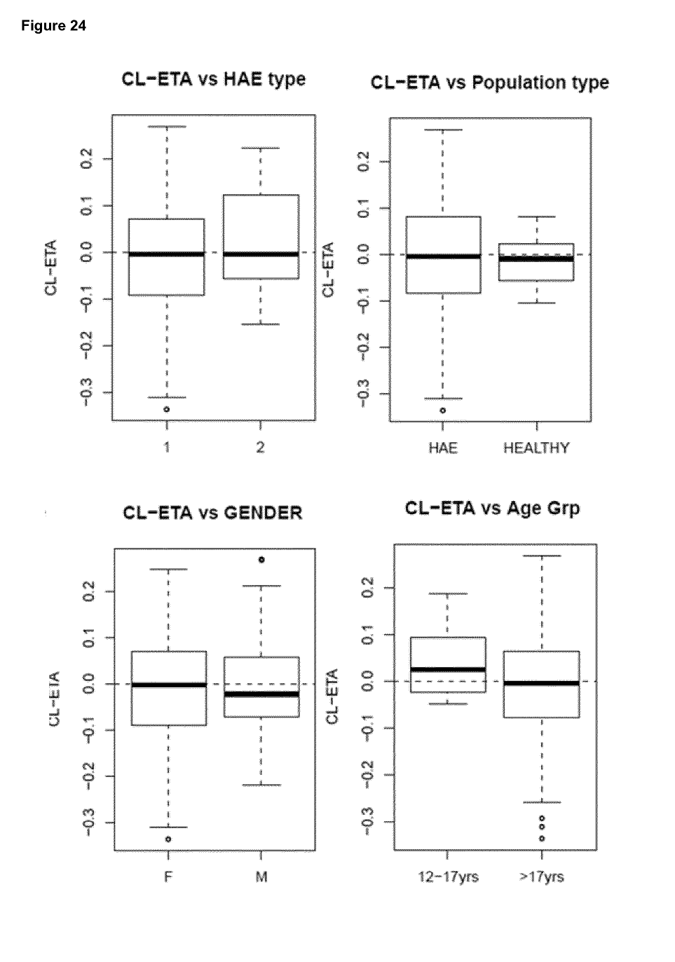

FIG. 24: ETA in CL vs. Covariate—Final Model (Run 012)

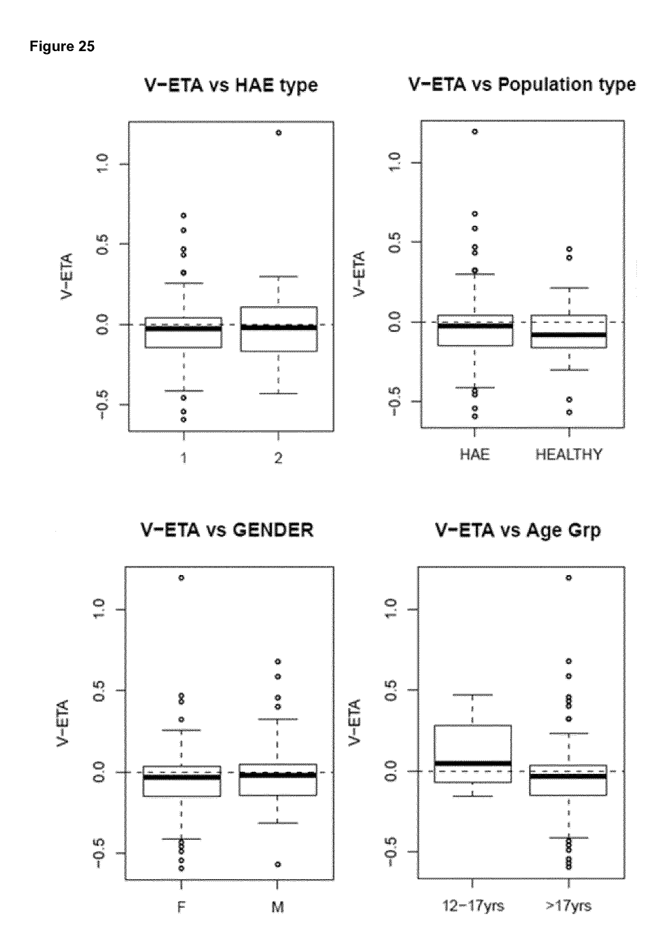

FIG. 25: ETA in V vs. Covariate—Final Model (Run 012)

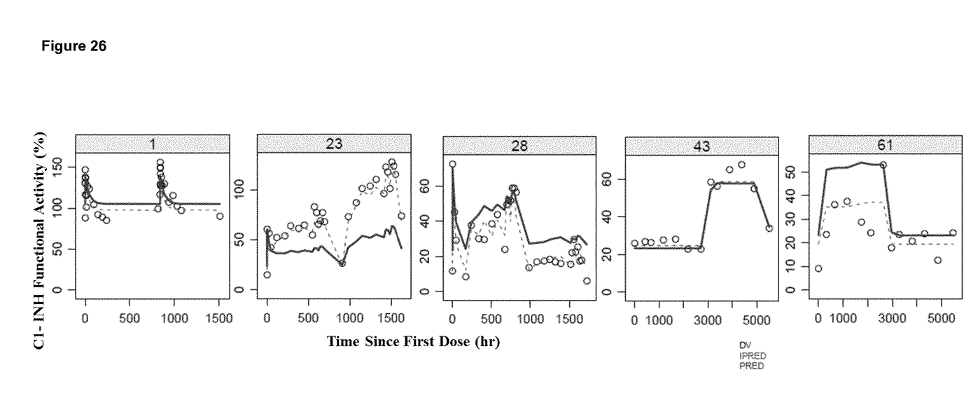

FIG. 26: Representative Individual Observed and Predicted Concentration—Final Model (Run 012)

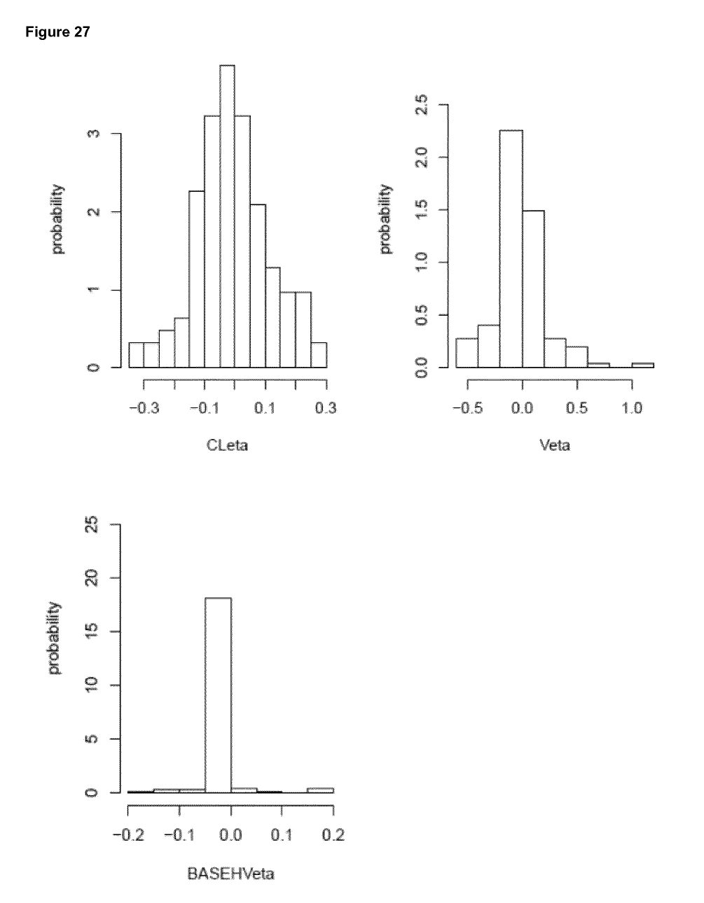

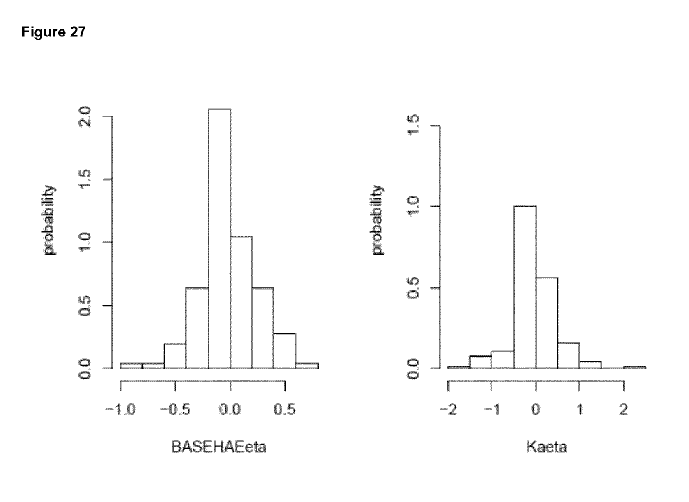

FIG. 27: Distributions of Interindividual Random Effects—Final Model (Run 012)

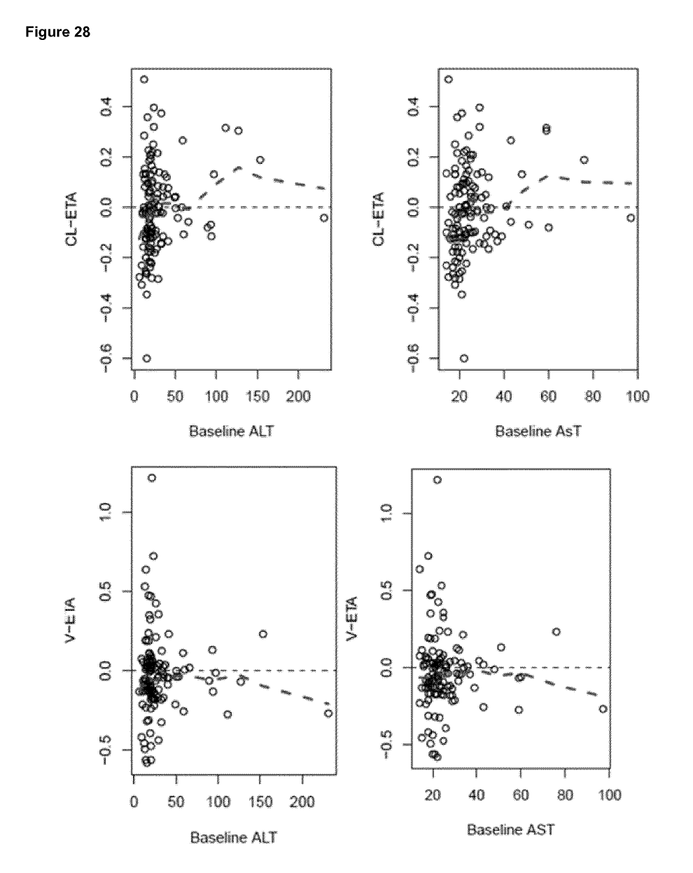

FIG. 28: Parameter ETA vs. Covariate plots—Base Model (008)

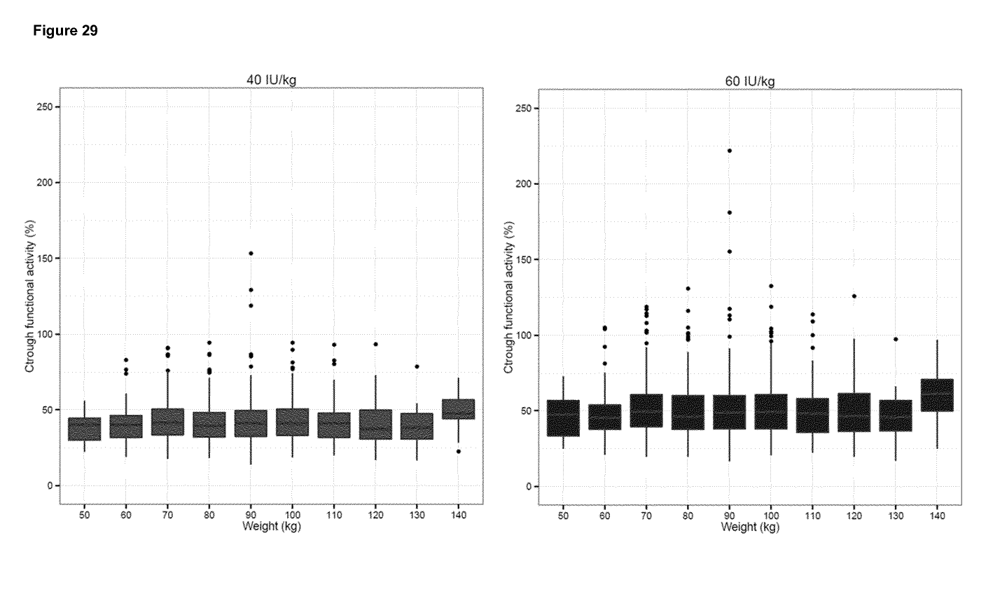

FIG. 29: Simulated Steady-state Trough C1-INH Functional Activity

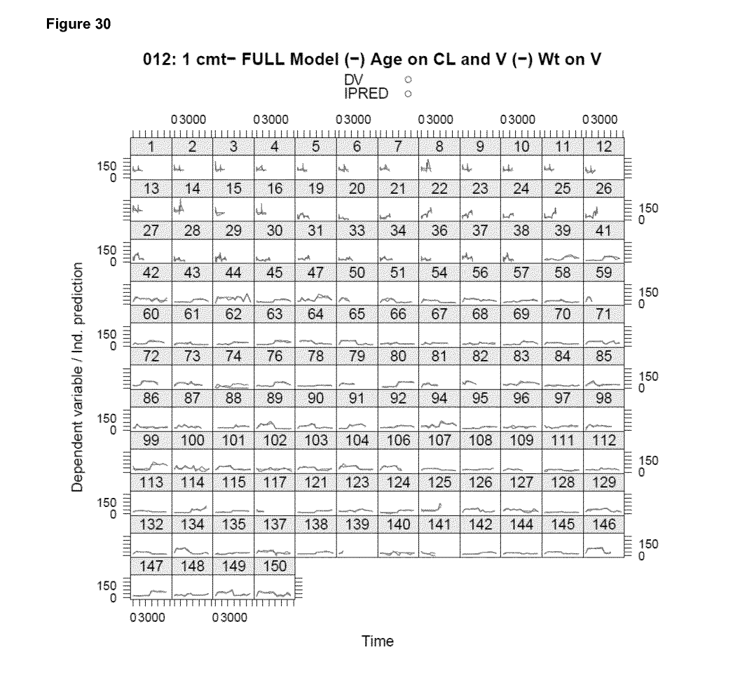

FIG. 30: Individual Observed and Predicted Concentration—Final Model (Run 012)

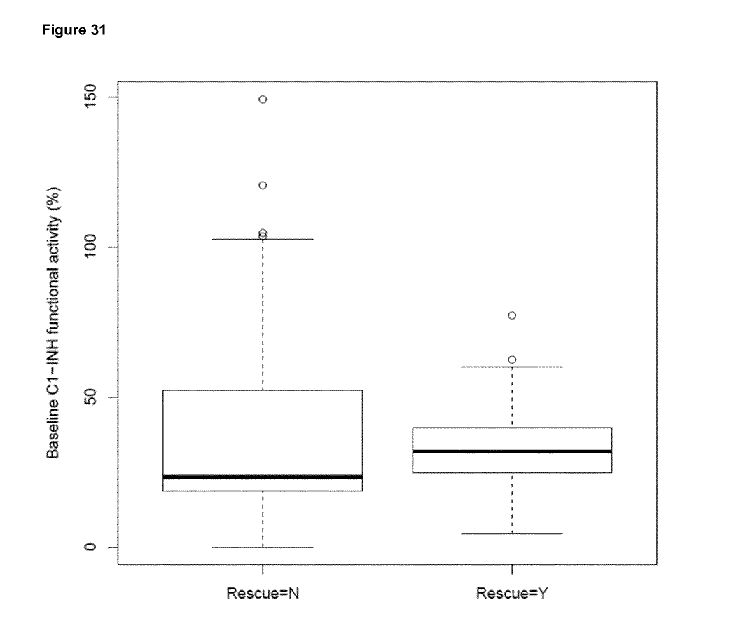

FIG. 31: Observed C1-INH Functional Activity vs. Patients Receiving Rescue C1-INH within 1 Week of Study

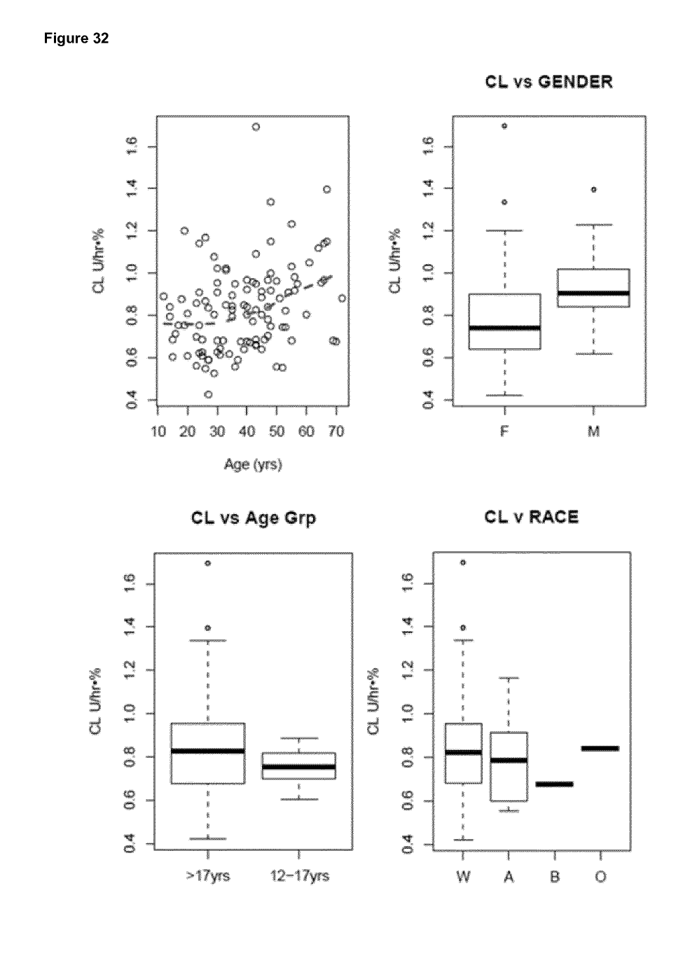

FIG. 32: Parameter CL vs. Covariate plots—Final Model (012)

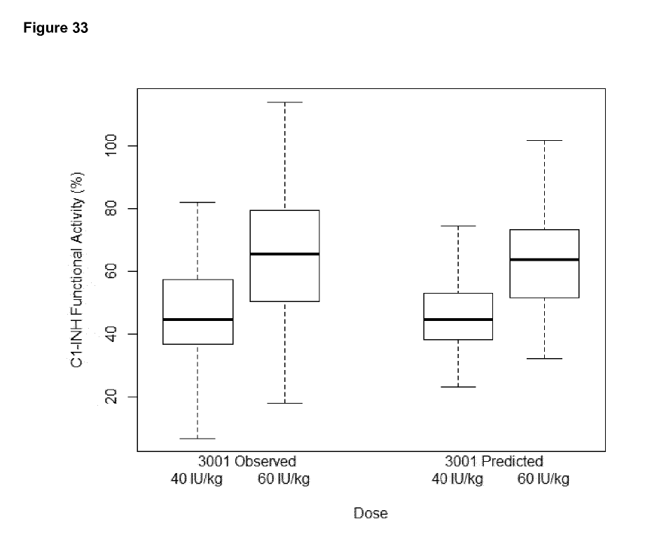

FIG. 33: Observed and Predicted Concentrations Stratified by Dose

### 5 LIST OF ATTACHMENTS

Attachment 1: Final Population Pharmacokinetic Output
Attachment 2: Modeling and Simulation Analysis Plan

### 6 INTRODUCTION

Hereditary angioedema (HAE) is a rare, autosomal dominant disorder characterized by clinical symptoms including edema, without urticaria or pruritus, generally affecting the subcutaneous (SC) tissues of the trunk, limbs, or face, or affecting the submucosal tissues of the respiratory, gastrointestinal, or genitourinary tracts [Agnosti and Cicardi, 1992; Davis, 1988]. Mutations in the SERPING1 gene encoding C1 esterase inhibitor (C1-INH) result in the most common types of HAE: C1-INH deficiency (HAE type 1; approximately 85% of affected individuals) and C1-INH dysfunction (HAE type 2; approximately 15% of affected individuals) [Bowen et al, 2010; Cugno et al, 2009; Davis 1988; Rosen et al, 1965].

Plasma-derived C1-INH administered intravenously (IV) is regarded as a safe and effective therapy for the management of patients with HAE [Zuraw et al, 2010], but a practical limitation of its long-term prophylactic use is the need for repeated IV access. Additionally, C1-INH functional activity levels tend to rapidly decline after IV administration of plasma-derived C1-INH. Routine IV prophylaxis with the approved 1000 IU dose (twice a week) results in recurrent periods of time when concentrations are likely to be sub-therapeutic and potentially associated the occurrence high rate of breakthrough attacks [Zuraw et al, 2015].

CSL Behring has developed CSL830, a high concentration, volume-reduced formulation of plasma-derived C1-INH for routine prophylaxis against HAE attacks by the subcutaneous (SC) route of administration. A previously conducted open-label, dose-ranging study (Study 2001) characterized the pharmacokinetics (PK)/pharmacodynamics (PD) and safety of SC administration of CSL830 in 18 subjects with HAE type 1 or 2. Subcutaneous administration of CSL830 increased trough C1-INH functional activity in a dose-dependent manner and was generally well-tolerated. A population PK analysis of the data from Study 2001 was conducted using a one-compartmental PK model with first-order absorption and first order elimination. The model provided a good description of the C1-INH functional activity-time data and revealed a significant effect of weight on the clearance (CL) of CSL830. Based on results from this model a body-weight based dosing regimen was for adopted for the pivotal study (Study 3001). Study 3001 was a Phase III, randomized, double-blind, placebo-controlled, incomplete crossover designed to assess the efficacy and safety of 2 doses of CSL830: 40 IU/kg (equivalent to 3000 IU for a 75 kg person) and 60 IU/kg (equivalent to 4500 IU for a 75 kg person). The study consisted of 2 consecutive treatment periods of up to 16 weeks each, during which subjects administered CSL830 or placebo at home twice per week in a double-blind, crossover manner.

The purpose of the current analysis is to characterize the population PK of C1-INH activity after administration of CSL830 in subjects with HAE, to identify covariates (demographic and clinical factors) that are potential determinants of C1-INH activity PK variability and to perform the simulations based on the final population model to support dosing of CSL830.

### 7 OBJECTIVES

The objectives of these analyses are:
To characterize the population PK of C1-INH functional activity in subjects with HAE
To identify sources of variability in C1-INH functional activity PK
To perform the simulations based on the final population model to support dosing of CSL830
To perform exploratory evaluation of the correlation between C1-INH activity, C1-INH antigen concentrations and C4 antigen concentrations

### 8 INVESTIGATIONAL PLAN

### 8.1 Study Population, Dose Regimens, and Pharmacokinetic Sampling

The population PK dataset consisted of data pooled from three clinical studies: Study 1001 titled “A randomized, double-blind, single-center, cross-over study to evaluate the safety, bioavailability and pharmacokinetics of two formulations of C1-esterase inhibitor administered intravenously; Study 2001 titled “An open-label, cross-over, dose-ranging study to evaluate the pharmacokinetics, pharmacodynamics and safety of subcutaneous administration of a human plasma-derived C1-esterase inhibitor in subjects with hereditary angioedema”; and Study 3001 titled “A double-blind, randomized, placebo-controlled, crossover study to evaluate the clinical efficacy and safety of subcutaneous administration of human plasma-derived C1-esterase inhibitor in the prophylactic treatment of hereditary angioedema”. In each study, PK was assessed using C1-INH functional activity in plasma and this was modeled in the current analysis. In addition, both C1-INH antigen and C4 antigen was measured and this data was assessed in an exploratory analysis. The PK population included subjects who received C1-INH either IV or SC and contributed at least one measurable PK concentration. A brief summary of the study characteristics are presented below and in Table 1.

### 8.1.1.1 Study 1001

Title: A randomized, double-blind, single-center, cross-over study to evaluate the safety, bioavailability and pharmacokinetics of two formulations of C1-esterase inhibitor administered intravenously.

This was a double-blind single dose PK and safety study in healthy volunteers to determine the relative bioavailability between IV administration of the established C1-INH formulation (50 IU human C1-INH per mL) and the concentrated formulation (CSL830; 500 IU human C1-INH per mL) that is in development for prophylactic SC administration for. The bioavailability of the two formulations was found to be comparable and safe to use in patients.

### 8.1.1.2 Study 2001

Title: An Open-label, Cross-over, Dose-ranging Study to Evaluate the Pharmacokinetics, Pharmacodynamics and Safety of the Subcutaneous Administration of a Human Plasma-derived C1-esterase Inhibitor in Subjects with Hereditary Angioedema.

This was an open label multiple dose PK study in HAE patients to determine the PK and PD of SC administration of 3 different dosing regimens of CSL830. Subjects were allocated sequentially to 1 of 6 possible CSL830 treatment sequences which was preceded by a single IV dose of C1-INH formulation currently on the market as treatment for acute attacks.

### 8.1.1.3 Study 3001

Title: A double-blind, randomized, placebo-controlled, cross-over study to evaluate the clinical efficacy and safety of subcutaneous administration of human plasma-derived C1-esterase inhibitor in the prophylactic treatment of hereditary angioedema.

This was a Phase III prospective double-blind placebo controlled study to investigate the clinical efficacy of SC administration of CSL830. In this study subjects were randomly assigned (1:1:1:1) to one of the 40 IU/kg CSL830 (sequences 1, 2) or 60 IU/kg CSL830 (sequences 3, 4) treatment sequences. Each sequence consisted of 2 consecutive periods (Treatment Period 1 and Treatment Period 2) of up to 16 weeks each. During the treatment periods, subjects administered CSL830 or placebo via SC injection twice a week in a double-blind cross-over manner. The detailed study design is available in the protocol.

TABLE 1
Summary of Studies Included in the Population PK Analysis

| | Population and | | |
|---|---|---|---|
| Study | No. Subjects | Dose/Treatment Duration | Planned PK Data |
| Study 1001 | 16 Healthy | Single dose of 1500 IU CSL830 or | C1-INH activity data after treatment with |
| (Phase I) | Volunteers | Berinert (50 IU/mL) given IV | both CSL830 and Berinert was used in the |
| | | | analysis. Intense PK samples were collected |
| | | | up to 24 hrs after dosing followed by |
| | | | intermittent samples until Day 11 after |
| | | | dosing. |
| Study 2001 | 18 HAE Patients | Single dose of 20 IU/kg Berinert | C1-INH activity data after treatment with |
| (Phase II) | | (50 IU/mL) followed by 1500 IU, 3000 IU | Berinert and various doses of CSL830 was |
| | | or 6000 IU of CSL830 given SC 2x per | used in the analysis. (Rescue C1-INH |
| | | week for 4 weeks | medication was also considered in the |
| | | | analysis). Intense PK samples were collected |
| | | | until 2 days after dosing followed by |
| | | | intermittent samples until the end of dosing |
| | | | at Week 4. |
| Study 3001 | 90 HAE Patients | 40 IU/kg or 60 IU/kg of CSL830 given | C1-INH activity data after treatment with |
| (Phase III) | | SC 2x per week for 16 weeks | various doses of CSL830 was used in the |
| | | | analysis. (Rescue C1-INH medication was |
| | | | also considered in the analysis). |
| | | | Sparse intermittent samples were collected |
| | | | throughout the study dosing at Week 16 in |
| | | | both periods of the study. |

### 8.2 Bioanalytical Methods

C1-INH functional activity was measured using a validated Berichrom C1-Inhibitor assay (Siemens Healthcare Diagnostics, Marburg, Germany).

The C1-INH functional activity, C1-INH antigen, and C4 antigen assays were validated with respect to accuracy, repeatability, precision, linearity, range, and robustness for determination of samples derived from clinical trials.

### 8.3 Data Retrieval

Subject data were collected in the case report form and were stored in the clinical database system by data management.

Data files containing all information for the modeling was provided to Eliassen Group (Wakefield Mass., USA) electronically in the form of SAS datasets, Excel spreadsheets, comma-separated ASCII files, or SAS transport files. Mapping documents were created to ensure traceability of each NONMEM input variable to its source in the original source datasets.

An error was discovered in the conversion factors used for fibrinogen test. Furthermore, the assignment for plasma-derived C1-INH prophylaxis or oral prophylaxis subgroups was updated. As a result the SDTM's and ADaM datasets were updated from the versions used in the creation of the original POPPK datasets. A comparison of the POPPK datasets based on the original sources files and of the updated source files demonstrated no significant difference. The details of the comparison are presented in the define package for the dataset.

### 8.4 DATA REVIEW

There were no data below the analytical assay quantification limit. Dosing events with missing dosing times were excluded from the analysis. If the exact dosing time for administration of rescue medication was missing, time 00:00 was used for the date of dosing. If covariate information (body weight, age) was missing at baseline, screening information was used. Screening values from screen failures were not used in this analysis.

### 8.5 Analysis Population

All subjects with evaluable dosing, actual sampling time, and concentration data were included in the analysis.

### 8.6 Pharmacokinetic Analyses Methods

Non-linear mixed effects modeling was performed using the computer program NONMEM version 7.2 (ICON Development Solutions, Ellicot City, Md., USA). For data presentation and construction of plots, Microsoft Excel, or R were used, as appropriate. PK parameters were estimated using the first-order conditional estimation method with interaction (FOCEI).

### 8.7 Population Pharmacokinetic Analysis

The population PK data in the subjects treated with CSL830 were analyzed using nonlinear-mixed effects modeling with NONMEM (v7.2), with the prediction of population pharmacokinetics (PREDPP) model library and NMTRAN subroutines. NONMEM runs were made on a grid of Linux servers. Analysis method using the methodology that imputes the measured plasma concentration values that are below limit of quantification [BLQ] to 0 was applied, only 2 values were BLQ in the analysis dataset. The first-order conditional estimation method with η-ε interaction (FOCE-INT) was employed for all runs. Perl speaks NONMEM (PsN) was used for Visual Predictive Check (VPC), and R version 3.1.1 (http://www.r-projector.org) was used for post-processing and plotting results. Data for rescue treatment during the study were included, whereas data prior to the start of Study 3001 were excluded from the analysis.

The analysis was conducted based on the following strategy:
Base Model Development,
Random Effect Model Development,
Inclusion of Covariates for Backward Elimination Approach,
Final Model Development,
Assessment of Model Adequacy (Goodness of Fit), and
Validation of the Final Model.

During model building, the goodness of fit of different models to the data were evaluated using the following criteria: change in the objective function, visual inspection of different scatter plots, precision of the parameter estimates, as well as decreases in both inter-individual variability and residual variability.

### 8.7.1.1 Base Model

The population PK models were developed by comparing 1- and 2-compartment models with first order elimination. The parameters of the models were expressed in terms of volume of distribution (Vd) and CL. For the PK models, endogenous C1-INH functional activity was modeled as an estimated parameter with a random effect. The observed C1-INH functional activity was the sum of the baseline values and the exogenous drug administered as shown below:

FTOT=F+BASE Equation 1

where FTOT=total plasma C1-INH functional activity estimate, F is the C1-INH functional activity due to CSL830 administration predicted from the model and BASE is the baseline C1-INH functional activity estimate. Model selection was driven by the data and was based on evaluation of goodness-of-fit plots (observed vs. predicted concentration, conditional weighted residual vs. predicted concentration or time, histograms of individual random effects, etc.), successful convergence (with at least 3 significant digits in parameter estimates), plausibility and precision of parameter estimates, and the minimum objective function value (OFV).

Distributions of individual parameters (Pᵢ) were assumed to be log-normal and were described by an exponential error model:

Pᵢ=TVP exp(η_{Pi}) Equation 2

where: Pᵢ is the parameter value for individual i, TVP is the typical population value of the parameter, and η_{Pi} are individual-specific inter-individual random effects for individual i and parameter P that are assumed to be normally distributed (η˜N(0, ω²)).

Model building was performed using diagonal covariance matrix of inter-individual random effects.

The residual error model was described by a proportional error model.

Y=F+F*ε Equation 3

where Y=dependent variable, F=prediction, ε=proportional residual error.

### 8.7.1.2 Covariate Modeling

The following covariates were considered before the start of the analysis: body weight, gender (Male=0, Female=1), age, HAE type, subject population (healthy or HAE patient), and region where the study was conducted.

Investigation of covariate-parameter relationships was based on the range of covariate values in the dataset, scientific interest, mechanistic plausibility, exploratory graphics and previously reported covariate-parameter relationships for CSL830 PK in other patient populations. Each covariate was evaluated individually. Insignificant or poorly estimated covariates (less than 10.84-point increase of OFV for one parameter, and/or confidence intervals include null value, and/or high relative standard error (RSE >50%)) were not included in the model. A full model approach was then implemented, where all covariate-parameter relationships that were thought to be significant were entered in the model, and parameters were estimated. Insignificant or poorly estimated covariates (less than 10.84-point increase of OFV for one parameter, and/or confidence intervals include null value, and/or high relative standard error (RSE >50%)) were then excluded from the model during the backward elimination process. Plots of eta-covariate values were reviewed after each major run to ensure all possible covariate-parameter relationships were evaluated.

For covariates to be explored in the analysis a continuous covariate had to have a sufficient range of values; categorical covariate had to be present in at least 10% of subjects in the data, unless there was a strong trend based on exploratory graphics suggesting potential influence of covariates on CSL830 PK. In these cases, the less prevalent covariates were also formally tested. In addition, only one of highly correlated covariates was allowed to enter the model at a time. For continuous covariates, a power function was utilized. For example:

TVPᵢ=θ₁*(COVᵢ/COV_{ST})^{θ}^{₂} Equation 4

where TVPᵢ is the typical value of a PK parameter (P) for an individual i with a COVᵢ value of the covariate, while θ₁ is the typical value for an individual with a standardized covariate value of COV_{ST}, and θ₂ is the influence of covariate on model parameter.

### 8.8 Model Evaluation and Discrimination

The goodness-of-fit (GoF) for a model was assessed by a variety of plots and computed metrics:
Observed versus population and individual predicted concentration plots;
Conditional weighted residuals (CWRES) versus population predicted concentrations and versus time plots;
Histograms of individual random effects to ensure they were centered at zero without obvious bias;
Scatter plots of individual random effects versus modelled covariates;
Relative standard errors (RSE) of the parameter estimates;
Shrinkage estimates for each η and ε,
Successful minimization and execution of a covariance step;
The minimum objective function value (OFV).

The difference in the objective function value (ΔOFV) between models was considered proportional to minus twice the log-likelihood of the model fit to the data and was used to compare competing hierarchical models. This ΔOFV was asymptomatically χ² distributed with degrees of freedom (d.f.) equal to the difference in number of estimated parameters between the two models. A ΔOFV with a χ² probability less than or equal to 0.01 (6.64 points of OFV, d.f.=1) would favor the model with the lower OFV. Backward elimination during covariate evaluation used a more stringent criterion at a significance level of less than or equal to 0.001 (10.84 points of OFV, d.f.=1).

### 8.9 Final Model Evaluation

### 8.9.1.1 Visual Predictive Check

The predictive performance of the final model was assessed by applying a posterior visual predictive check (VPC) [Yano et al, 2001]. The final model was used to simulate 1000 datasets based on the covariates, sampling times and the dosing histories contained in the dataset. The original dataset was compared with the 5^{th}, 10^{th}, 90^{th}, and 95^{th} percentiles for the simulated data for each time. The number of observed concentrations that fell within the 80% and 90% prediction intervals was determined by population type (HAE vs. HV). This comparison was used to evaluate whether the derived model and associated parameters were consistent with the observed data.

### 8.9.1.2 Bootstrap Analysis

In addition to the VPC, the final PK model was subjected to a nonparametric bootstrap analysis, generating 1000 datasets through random sampling with replacement from the original data using the individual as the sampling unit. Population parameters of the final PK model for each dataset were estimated using NONMEM. This resulted in a distribution of estimates for each population model parameter. Empirical 95% confidence intervals (CI) were constructed by obtaining the 2.5^{th} and 97.5^{th} percentiles of the resulting parameter distributions. Estimates from all NONMEM runs (with successful and unsuccessful minimization) were reported.

### 8.10 Simulations

The final model was used to simulate plasma functional activity profiles for the treatment-experienced population.

C1-INH functional activity was predicted from first dose up to steady-state achieved following a 40 IU/kg or 60 IU/kg twice weekly dose of CSL830. In this procedure, parameters obtained from the population model were used to simulate 1000 individual profiles based on the distribution of individual weights from the population PK analysis.

### 8.10.1.1 Individual Predicted Pharmacokinetic Parameters

Concentration-time profiles (concentrations simulated at Day 1-Day 8) following a steady-state dose of CSL830, for respective individuals using their individual parameter values and dosing regimen, were simulated for each dose assuming zero values for residual variability. The individual estimates of all model parameters were obtained from the final model by an empirical Bayes estimation method. Individual estimates of AUC_{0-τ} were be calculated as

$${AUC}_{0 - τ} = \frac{Dose ⋆ {F}_{i}}{{CL}_{i}} \qquad Equation 5$$

Where: AUC_{0-τ} was area under the curve at steady state during a dosing interval (patients were dosed twice a week), Dose was amount received by each subject, CLᵢ was the individual estimate of clearance, and Fᵢ was the individual estimate of relative s.c. bioavailability. Individual estimates of C_{avg} were calculated as

$${C}_{avg} = \frac{{AUC}_{0 - 168}}{168} \qquad Equation 6$$

Where: AUC₀₋₁₆₈ was area under curve at steady state during a week (168 hrs). The AUC₀₋₁₆₈ was used since the patients were dosed twice a week, the exposures during the week provided more accurate estimates of the C_{avg}. Individual steady state estimates of Cₘₐₓ, C_{trough}, Tₘₐₓ, half-life and apparent half-life were computed for each individual. The half-life was calculated as

$${t}_{1 / 2} = \frac{ln ( 2 )}{{CL}_{i} / {V}_{i}} \qquad Equation 7$$

Where: CLᵢ was the individual estimate of clearance and V, was the individual estimate of volume of distribution. Apparent half-life was calculated from the terminal slope of the C1-INH functional activity profiles. Summary statistics (geometric mean, CV %, 95% CI, median, range and percentiles (5%, 10%, 25%, 75%, 90% and 95%)) for AUC_{0-τ}, Cₘₐₓ, Tₘₐₓ and half-life and C_{trough} were computed for each dose.

### 9 RESULTS

### 9.1 Dataset Analyzed

A total of 124 subjects (108 HAE and 16 Healthy Volunteers) from Studies 1001, 2001, and 3001 were included in the PK analysis dataset. The dataset included 2103 C1-INH functional activity observations. The observed C1-INH functional activity over time stratified by study is presented in FIG. 9.

### 9.2 Demographics and Covariates

The demographics of this population by study are summarized in Table 2. The number of non-Caucasian subjects in the study account for <10% of the population and the covariate of race was therefore considered unsuitable to be included in the covariate analysis.

TABLE 2
Subject Characteristics and Demographics by Study

| | Statistic or | | | | |
|---|---|---|---|---|---|
| Covariate | category | Study 1001 | Study 2001 | Study 3001 | Overall |
| Total Number | | | | | |
| Age (yrs) at baseline | Median [Min-Max] | 35.0 [24-45] | 33.5 [18-69] | 40.0 [12-72] | 38.5 [12-72] |
| Weight (kg) at baseline | Median [Min-Max] | 73.7 [54-108] | 78.9 [51-110] | 78.1 [43-157] | 77.6 [43-157] |
| Observed Baseline C1-INH | Mean [Min-Max] | 99.8 [79-149] | 17.9 [0-43] | 28.6 [4.5-77] | 36.5 [0-149] |
| functional activity | | | | | |
| Gender N | Male | 11 | 7 | 30 | 48 |
| | Female | 5 | 11 | 60 | 76 |
| Race N | Caucasian | 16 | 14 | 84 | 114 |
| | Asian | — | 4 | 4 | 8 |
| | Black | — | — | 1 | 1 |
| | Other | — | — | 1 | 1 |
| HAE Type N | Type 1 | | 16 | 78 | 94 |
| | Type 2 | NA | 2 | 12 | 14 |
| Total No. of samples N | | 496 | 545 | 1062 | 2103 |

### 9.3 Base Model Development

CSL830 functional activity was best described by a one-compartment model with first order absorption when administered SC with structural parameters for CL and Vd, first order absorption rate constant (ka), and baseline C1-INH functional activity. A two-compartment model with first order absorption was also fitted to the data. Based on model diagnostics, the one-compartment model provided better description of the data. The baseline C1-INH functional activity is unambiguously different (FIG. 10) between patients and healthy subjects due to the nature of the disease state. To account for this difference, separate baseline parameters were estimated for each population.

The parameter estimates from the base model are listed in Table 3. The population mean for bioavailability of subcutaneously administered CSL830 was fixed to the value obtained from the population PK analysis from Study 2001 [Zuraw et al, 2015]. The parameters were estimated with good precision as indicated by low % RSE (<20%).

TABLE 3
Parameter Estimates of Base CSL830 Population PK Model

| Parameter | NONMEM Estimates | | | |
|---|---|---|---|---|
| [Units] | Point Estimate | % RSE | IIV % | % RSE |
| CL [IU/hr · %] | 0.839 | 6.71 | 30.6 | 19.8 |
| Vd [IU/%] | 43.5 | 9.00 | 40.7 | 31.1 |
| Ka [hr⁻¹] | 0.0142 | 12.6 | 80.4 | 13.9 |
| BASE | 106 | 3.18 | 11.0 | 18.3 |
| [%](Healthy | | | | |
| volunteers)[hr] | 23.3 | 3.62 | 29.7 | 10.0 |
| BASE [%] | | | | |
| (HAE patients) | | | | |
| F | 0.427 | FIX | 54.0 | 12.1 |
| Residual variability | | CV % | | % RSE |
| σ² prop | | 23.4 | | 5.0 |

Abbreviations:
% RSE: percent relative standard error of the estimate = SE/parameter estimate * 100, 95%,
CL = clearance,
Vd = volume of central compartment,
Ka = absorption rate constant,
CV = coefficient of variation of proportional error (=[σ² prop]^{0.5} * 100),
σ² prop = proportional component of the residual error model.
IIV = inter individual variability (=[σ² prop]^{0.5} * 100)

Diagnostic plots (FIG. 11) did not reveal any major concerns with the fit and demonstrated good agreement between predicted and observed data.

### 9.4 Covariate Model Development

The relationships between covariates of interest and the predicted etas for both CL and Vd were explored visually (FIG. 12). Based on this visual inspection and clinical interest, the covariates tested included age, and body weight at baseline on CL and age and body weight at baseline on Vd being added simultaneously to form a full model. The reference covariate value used in the model was 80.7 kg for body weight (mean) and 38.5 years for age (median). Body weight on CL was the only covariate that was found to be statistically significant. The key analysis steps of the backward elimination process for covariate testing are provided in Table 4.

TABLE 4
Summary of Covariate Model Development

| Run | | Reference | | OFV | Minimization | Covariance |
|---|---|---|---|---|---|---|
| No | Model Description ^{a} | Model | OFV | Change | (Y/N) | (Y/N) |
| 008 | 1 compartment model with Ka, CL, V, BASE | — | 13355 | — | Y | Y |
| | for HAE and HV, F, eta (CL, V, Ka, BASE | | | | | |
| | for HAE, BASE for HV, F), proportional | | | | | |
| | residual error model; | | | | | |
| | [Base model] | | | | | |
| 010 | Add Age and Wt on CL and V [Full model] | 008 | 13332 | −23.40 | Y | Y |
| 009 | Remove Age on V | 010 | 13332 | 0 | Y | Y |
| 011 | Remove Age on CL | 009 | 13332 | 0.075 | Y | Y |
| *012 | Remove Wt on V | 011 | 13336 | 3.71 | Y | Y |
| 013 | Remove Wt on CL [Base model] | 012 | 13355 | 19.6 | Y | Y |
| 017 | Add Study 2001 as covariate on CL | 012 | 13315 | −20.3 | Y | Y |
| 019 | Include Rescue medication before start of | 012 | 13298 | −37.5 | N | N |
| | study | | | | | |
| 040 | 2 compartment model with Ka, CL, V, BASE | | | | | |
| | for HAE and HV, F, eta (CL, V, Ka, BASE | 001 | 13484 | — | Y | N |
| | for HAE, BASE for HV), proportional | | | | | |
| | residual error model; | | | | | |

^{a}. CSL830_1001_2001_3001_POPPK_24JAN2016.csv was used for all models
b. Abbreviations: CL = total clearance, BASE: Baseline C1-INH functional activity, V = Volume of distribution, Ka = absorption rate constant, WT: body weight
*Final model

### 9.5 Final Model

The final population PK model had only one covariate effect: bodyweight on CL. Table 5 compares the final PK parameter estimates with the median and 95% CIs derived from the bootstrap runs.

The estimates of CL, Vd, Ka, BASE were consistent with the results from the previously conducted population PK analysis. The final CSL830 population PK model equation for CL:

$$CL = 0.830 ⋆ {( \frac{WT}{80.5} )}^{0.738} \qquad Equation 8$$

TABLE 5
Parameter Estimates of Final CSL830 Population PK Model

| Parameter | NONMEM Estimates | | | Bootstrap Estimates^{a} | | |
|---|---|---|---|---|---|---|
| [Units] | Point Estimate | % RSE | % IIV | % RSE | Median | 95% CI |
| CL [IU/hr · %] | 0.830 | 6.40 | 24.2 | 22.9 | 0.830 | 0.727-0.942 |
| Vd [IU/%] | 43.3 | 9.60 | 39.2 | 32.2 | 42.4 | 35.1-51.5 |
| Ka [hr⁻¹] | 0.0146 | 16.1 | 82.2 | 14.5 | 0.0143 | 0.0109-0.0194 |
| BASE [%](Healthy | 105 | 3.20 | 11.03 | 17.8 | 105 | 98.7-113 |
| volunteers)[hr] | | | | | | |
| BASE [%] (HAE | 23.2 | 3.68 | 29.5 | 9.76 | 23.3 | 21.5-24.9 |
| patients) | | | | | | |
| F | 0.427 | FIX | 49.1 | 12.6 | 0.427 | NA |
| Effect of Body | 0.738 | 23.8 | | | 0.731 | 0.403-1.07 |
| weight on CL | | | | | | |

Inter-individual or inter-occasion variability

| | | | |
|---|---|---|---|
| ω_{CL}² | 0.0587 | 0.054 | 0.0148-0.134 |
| ω_{V}² | 0.153 | 0.135 | 6.4E−07-0.379 |
| ω_{BASE HV}² | 0.0122 | 0.0106 | 0.00304-0.0204 |
| ω_{BASE HAE}² | 0.0868 | 0.0862 | 0.0572-0.129 |
| ω_{Ka}² | 0.675 | 0.635 | 0.0453-1.104 |
| ω_{F}² | 0.241 | 0.243 | 0.130-0.374 |

| Residual variability | CV % | % RSE |
|---|---|---|
| σₚᵣₒₚ² | 23.4 | 5.10 |

^{a}From 1000 bootstrap runs.
Abbreviations: % RSE: percent relative standard error of the estimate = SE/parameter estimate * 100, 95% CI = 95% confidence interval on the parameter, CL = clearance, V = volume of central compartment, Ka = absorption rate constant, ω_{CL}² = variance of random effect of CL, CV = coefficient of variation of proportional error (=[σₚᵣₒₚ²]^{0.5}*100), σₚᵣₒₚ² = proportional component of the residual error model, WT = baseline weight (kg).

Diagnostic plots (FIG. 14) did not reveal any major concerns with the fit. The shrinkage estimate for CL was 50%, and for Vd was 40%.

There was a clear relationship between CL and body weight observed in the base model (FIG. 12). This relationship is accounted for in the final model by the inclusion of body weight as a covariate on CL as evidenced in FIG. 15 (i.e. etas are well centered on the mean of zero).

### 9.6 Final Model Evaluation

The final model was evaluated by visual predictive checks. The final model population parameters and inter-individual error estimates were used to simulate concentrations back into the observed datasets using PsN. Simulations with the final model and parameter estimates were conducted for 1000 individuals. The observed concentrations for healthy volunteers and HAE patients at 10^{th} and 90^{th} percentiles and median were inspected for agreement with simulated concentrations at the 10^{th}, 50^{th}, and 90^{th} percentiles. Visual predictive checks for the final population PK model are shown in FIG. 16. Overall, these diagnostic plots do not indicate any substantive deficiency in the ability of the final reference model to characterize the trend and variability in the observed PK data.

### 9.7 Posthoc Analysis

Visual evaluation of individual post-hoc CL estimates revealed that the CL was lower in patients enrolled in Study 2001 when compared to Study 3001. This was quantified in the final model as a categorical covariate and the CL was estimated to be 40% lower in patients enrolled in Study 2001. The individual post-hoc CL and Vd estimates from the two models showed no difference. Hence, the final model did not include Study 2001 as a covariate (FIG. 17).

Visual evaluation of individual observed baseline C1-INH functional activity revealed that the distribution of the baseline values was similar between patients that received IV C1-INH as rescue mediation for HAE attacks within 1 week of start of study compared to the patients that did not receive IV C1-INH as rescue mediation within 1 week of start of the study. The median of the two groups was slightly different, that can be due to the different sample sizes. The model accounting for the IV C1-INH as rescue mediation for HAE attack before the start of the study was unable to convergence and minimize successfully. This could be due to lack of observed data during this period. Hence, the final model did not include information regarding IV C1-INH as rescue mediation for HAE attack before start of the study.

### 9.8 Simulations

C1-INH functional activity versus time profiles after 4 weeks of twice weekly dosing of 40 IU/kg or 60 IU/kg CSL830 (doses used in Phase 3; Study 3001) were simulated in 1000 HAE patients using the final model. The median (90% CI) simulated C1-INH functional activity time curve are presented in FIG. 18.

The simulated steady-state geometric mean of maximum functional activity (Cₘₐₓ) was 48.7%, and the minimum functional activity (C_{trough}) at steady state was 40.2% for 40 IU/kg dose and Cₘₐₓ was 60.7%, and C_{trough} was 48.0% for 60 IU/kg dose. A summary of the model-predicted Cₘₐₓ, C_{trough}, C_{avg} and AUC_{0-τ} are presented in Table 6.

TABLE 6
Summary of Steady-State CSL830 C ₘₐₓ, Cₘᵢₙand AUC_{0-ττ}from the Simulated Population
Stratified by Dose

| | | | | | | * (hr) Apparent | |
|---|---|---|---|---|---|---|---|
| | | | | | | Half-Life *^{†} | |
| Dose C | ₘₐₓ (%) T | ₘₐₓ* (hr) | AUC_{0-ττ}(%*h) | Ctrough (%) C | _{avg} Half-life | (hr) | |
| 40 IU/kg | 48.7 | 58.7 | 1700 | 40.2 | 44.6 | 36.9 | 68.7 |
| | (26.9-96.2) | (23-134) | (558-5110) | (22.2-77.9) | (24.7-86.3) | (14.3-102) | (24.0-250) |
| 60 IU/kg | 60.7 | 58.7 | 2540 | 48.0 | 54.8 | 36.9 | 68.7 |
| | (31.8-128) | (23-134) | (837-7670) | (25.1-102) | (29.2-112) | (14.3-102) | (24.0-251) |

Data presented as geometric mean (95% CI)
*Data presented as Median (95% CI)
^{†}Calculated using NCA module in Phoenix ©

### 9.9 Exploratory Analysis

In addition to the measurement of C1-INH functional activity, both the C1-INH antigen (collected in Studies 1001, 2001, and 3001) and C4 antigen (collected in Studies 2001 and 3001) were also collected in the clinical program. The relationships between C1-INH functional activity and these antigens were visually inspected in an exploratory manner. Five subjects in the dataset were classified as HAE type 2 despite their C1-INH antigen levels below 0.2 mg/mL at screening. These patients were excluded from the exploratory biomarker analysis.

### 9.9.1.1 C1-INH Antigen

FIG. 19 represents C1-INH antigen concentrations vs. time after dose in each study. The C1-INH antigen concentrations appear to increase after CSL830 administration and then decrease over time.

FIG. 20 presents the relationship between C1-INH antigen and C1-INH functional activity. The relationship appears to be linear up to a C1-functional activity level of ˜150 at which point the loess fit appears to reveal signs of saturability. In patients with HAE type 1 (C1-INH antigen deficient), a linear relationship is apparent across the range of antigen and functional activity levels observed in the clinical program. In patients with HAE type 2 (dysfunctional C1-INH), a linear relationship is apparent in Study 2001 study, however the relationship is not clearly evident in the Study 3001 study, potentially due to the limited number of data points.

### 9.9.1.2 C4 Antigen

FIG. 21 presents C4 antigen concentrations vs. time after dose, stratified by study. The C4 antigen concentrations appear to increase after CSL830 administration and then decrease over time (after ˜100 hrs).

FIG. 22 presents the relationship between C4 antigen and C1-INH functional activity in HAE patients. The relationship appears to be linear in HAE type 1 subjects, up to a C1-INH functional activity level of ˜50, at which point the Loess fit appears to reveal signs of saturability. The relationship is not clearly evident in subjects with HAE type 2, potentially due to the limited number of data points.

9.9.1.3 C1-INH Antigen vs. C4 Antigen

FIG. 23 presents the relationship between C4 antigen and C1-INH antigen concentrations. The relationship appears to be a linear up to C1-INH antigen concentrations of ˜0.1 mg/mL at which point the C4 antigen concentrations are approaching the normal range.

### 10 DISCUSSION

The objectives of this analysis were to describe the PK of C1-INH functional activity after administration of CSL830 to HAE patients and to estimate the effects of covariates on the variability of these PK parameters using data from three clinical studies (Studies 1001, 2001, and 3001). Studies 1001 and 2001 employed fixed doses whereas Study 3001 employed weight based dosing. In addition, patients in Studies 2001 and 3001 were allowed the use of IV C1-INH as rescue mediation for HAE attacks and these records were included in the model.

A one-compartment model with first-order absorption and first order elimination described the structure of the PK model for C1-INH functional activity. Since HAE is a disease resulting from a deficiency in C1-INH functional activity, separate baseline parameters were included in the model for HAE patients (Studies 2001 and 3001) and healthy volunteers (Study 1001). The bioavailability of CSL830 was fixed at 0.43, which was estimated in Study 2001. Study 2001 included patients treated with both IV and SC administration of CSL830 and hence allowed the ability to accurately estimate the bioavailability. A backward elimination approach was employed to test covariates of interest including body weight, and age on CL and Vd. The results of the covariate testing indicated weight is significant covariate on CL. Weight was not a significant covariate on Vd, and age was not a significant covariate on CL or Vd. Visual inspection did not elucidate a difference in PK parameters between male and female or between regions where the study was conducted. Race as a covariate was not tested as the Caucasian population constituted greater than 90% of the data.

The final model provided a good description of the C1-INH functional activity data in healthy volunteers and HAE patients. Goodness-of-fit criteria, revealed that the final model was consistent with the observed data and that no systematic bias remained. The allometric exponent of weight on CL was estimated to be 0.74, which is similar to the theoretical value of 0.75. To illustrate the magnitude of this effect, a subject with a baseline weight of 60 kg would have a CL of 0.67 IU/hr·%, whereas a subject with a baseline weight on 90 kg would have a CL of 0.90 IU/hr·%.

The PK parameter estimates from the analysis provided in this report are different when compared to the model developed based on the Study 2001 study alone [Zuraw et al, 2015]. The lower CL estimates in Study 2001 compared to Study 3001 could be due to the smaller sample size in Study 2001 or due to the higher rate of HAE attacks prior to screening in Study 3001, which may have an impact on the CL of CSL830. It is believed that during an HAE attack a considerable amount of C1-INH is consumed by the patient, which may increase the CL of C1-INH functional activity; however this has not been published in the literature. The population mean F, CL and Vd obtained from the current analysis for C1-INH are consistent with NCA estimates as reported in the literature [Martinez-Sauger et al, 2010; Hofstra et al, 2012; Martinez-Sauger et al, 2014].

NCA could not be employed with the data from this study due to a) the limited number of PK samples collected and b) the use of rescue medication which can have a confounding effect on the observed C1-INH functional activity. The population PK model developed in this analysis allowed the ability to estimate key PK parameters of CSL830. Based on the final model, mean Cₘₐₓ was 48.7% for 40 IU/kg, and 60.7% for 60 IU/kg, and mean C_{trough} was 40.2% for 40 IU/kg, and 48.0% for 60 IU/kg. Weight-based dosing presents less population variability of simulated trough activity levels (FIG. 29). From the final model, the Tₘₐₓ for CSL830 was 58.7 hours (˜2.5 days) and half-life was 36.9 hours. The Tₘₐₓ of ˜2.5 days is characteristic of subcutaneous administration of proteins. The calculated half-life estimates were consistent with parameter estimates in HAE patients from prior C1-INH functional activity studies [Martinez-Sauger et al, 2010; Kunschak et al, 1998].

An exploratory analysis demonstrated a linear relationship between C1-INH functional activity and C1-INH antigen. A similar relationship is observed between C1-INH functional activity and C4 antigen. The observed relationships between C4 antigen and C1-INH antigen/functional activity in this analysis are consistent with previous reports [Spath et al, 1984].

Current practice includes assessment of C1-INH functional activity as a biomarker of HAE. The clinical utility of monitoring C4 or C1-INH antigen is unknown. The interplay between C1-INH functional activity, C1-INH antigen and C4 antigen can be should be further explored to make decisions regarding dose-adjustments in patients with suboptimal protection from HAE attacks.

### 11 CONCLUSIONS

C1-INH functional activity was well described by a one-compartment model with first order absorption.

Body weight was a significant covariate that affected CL of CSL830.

Simulations at 40 IU/kg and 60 IU/kg twice weekly dose of CSL830 results in a mean C_{trough} of 40.2 and 48.0% C1-INH functional activity respectively.

### 12 QUALITY CONTROL

The Population PK report was subject to scientific review and quality control (QC) according to CSL template PK-TPL-03.

### 13 REFERENCES

Agostoni A, Cicardi M. Hereditary and acquired C1-inhibitor deficiency: biological and clinical characteristics in 235 patients. Medicine (Baltimore) 1992; 71(4):206-15.
Bork K. Human pasteurized C1-inhibitor concentrate for the treatment of hereditary angioedema due to C1-inhibitor deficiency. Expert Review of Clinical Immunology 2011; 7(6):723-733.
Bowen T, Cicardi M, Farkas H, et al. 2010 international consensus algorithm for the diagnosis, therapy and management of hereditary angioedema. Allergy Asthma Clin Immunol 2010; 6:24.
Cugno M, Zanichelli A, Foieni F, et al. C1-inhibitor deficiency and angioedema: molecular mechanisms and clinical progress. Trends Mol Med 2009; 15:69-78.
Davis A E, III. C1 inhibitor and hereditary angioneurotic edema. Annu Rev Immunol 1988; 6:595-628.
European Medicines Agency. Guideline on Reporting the Results of Population Pharmacokinetic Analyses. 2007.
Hofstra J J, Kleine Budde I, van Twuyver E, et al. Treatment of hereditary angioadema with nanofiltered C1-esterase inhibitor concentrate (Cetor®): multi-center phase II and III studies to assess pharmacokinetics, clinical efficacy and safety. Clin Immunol 2012; 142(3):280-90.
Kunschak M, Engl W, Maritsch F, et al. A randomized, controlled trial to study the efficacy and safety of C1 inhibitor concentrate in treating hereditary angioedema. Transfusion 1998; 38:540-9.
Martinez-Sauger I, Rusicke E, Aygoren-Pursun E, et al. Pharmacokinetic analysis of human plasma-derived pasteurized C1-inhibitor concentrate in adults and children with hereditary angioedema: a prospective study. Transfusion 2010; 50(2):354-60.
Martinez-Sauger I, Cicardi M, Suffritti C, et al. Pharmacokinetics of plasma-derived C1-esterase inhibitor after subcutaneous versus intravenous administration in subjects with mild or moderate hereditary angioedema: the PASSION study. Transfusion 2014; 54: 1552-61.
Rosen F S, Pensky J, Donaldson V, Charache P. Hereditary angioneurotic edema: two genetic variants. Science 1965; 148:957-58.
Späth P J, Wüthrich B, Bütler R. Quantification of C1-inhibitor functional activities by immunodiffusion assay in plasma of patients with hereditary angioedema—evidence of a functionally critical level of C1-inhibitor concentration. Complement 1984; 1(3):147-159.
US Food and Drug Administration. Guidance for Industry: Population Pharmacokinetics. 1999.
Yano Y, Beal S L, Sheiner L B. Evaluating pharmacokinetic/pharmacodynamic models using the posterior predictive check. J Pharmacokinet Pharmacodyn 2001; 28(2): 171-92.
Zuraw B L. Diagnosis and management of hereditary angioedema: an American approach. Transfusion and Apheresis Science 2003; 29(3): 239-45.
Zuraw B L, Busse P J, White M, et al. Nanofiltered C1 inhibitor concentrate for treatment of hereditary angioedema. N Engl J Med 2010; 363(6):513-522.
Zuraw B L, Cicardi M, Longhurst H J, et al. Phase II study results of a replacement therapy for hereditary angioedema with subcutaneous C1-inhibitor concentrate. Allergy 2015; 70(10):1319-28.

1-54. (canceled)

55. A method of treating hereditary angioedema and/or of preventing hereditary angioedema attacks, comprising administering C1-INH to a patient according to a dosing scheme, wherein the dosing scheme for C1-INH is based on administration of a therapeutic C1-INH concentration (Cp), wherein the Cp is determined using an age-dependent risk-for-an-angioedema-attack model, and wherein the C1-INH dosing maintains a trough level C1-INH functional activity above Cp.

56. The method of claim 55, wherein the model involves the parameters
(i) background risk (B0),
(ii) effect of patient age on background risk (Age on B0),
(iii) maximum C1-INH effect (Eₘₐₓ), and
(iv) half maximal effective concentration of C1-INH (EC₅₀).

57. The method of claim 55, wherein the model is based on formula
$$h = {e}^{BO} \times {( \frac{age}{42} )}^{Age on B 0} \times e ( ( E max ) \times \frac{Cp}{( {e}^{EC 50} + Cp )} )$$
wherein h is the risk for an attack and age is the individual patient's age.

58. The method of claim 56, wherein
(i) B0 ranges from about −0.665 to 0.825,
(ii) Age on B0 ranges from about 0.552 to 1.55,
(iii) Eₘₐₓ ranges from about −11.2 to −9.84, and/or
(iv) EC₅₀ ranges from about 3.16 to 3.64.

59. The method of claim 56, wherein
(i) B0 is about 0.0802,
(ii) Age on B0 is about 1.05,
(iii) Eₘₐₓ is about −10.5,
and/or
(iv) EC₅₀ is about 3.4.

60. The method of claim 55, wherein the risk of occurrence of an angioedema attack is selected to result in equal or less than one attack per month.

61. The method of claim 55, wherein the risk of occurrence of an angioedema attack is selected to result in equal or less than one attack per year.

62. The method of claim 55, wherein the C1-INH dosing scheme is determined using a one-compartmental pharmacokinetics model with first order absorption and first order elimination.

63. The method of claim 62, wherein the one-compartmental pharmacokinetics model is weight-dependent.

64. The method of claim 55, wherein the C1-INH is administered via subcutaneous administration.

65. The method of claim 55, wherein the patient self-administers C1-INH.

66. The method of claim 55, wherein the C1-INH is derived from human plasma.

67. The method of claim 55, wherein the hereditary angioedema is type 1 hereditary angioedema or type 2 hereditary angioedema.

68. A computer usable medium comprising computer-executable instructions for determining a therapeutic C1-INH concentration (Cp), comprising: means for causing a computer to determine a Cp for the treatment of hereditary angioedema and/or the prevention of hereditary angioedema attacks in an individual patient using an age-dependent risk-for-an-angioedema-attack model.

69. A computer comprising the computer program product of claim 68.

70. A device for determining a C1-INH dosing scheme for the treatment of hereditary angioedema and/or the prevention of hereditary angioedema attacks in an individual patient, comprising:
(i) A computer usable medium comprising computer-executable instructions for determining a therapeutic C1-INH concentration (Cp), comprising: means for causing a computer to determine a Cp for the treatment of hereditary angioedema and/or the prevention of hereditary angioedema attacks in an individual patient using an age-dependent risk-for-an-angioedema-attack model, and
(ii) a computer capable of executing the instructions.

71. A kit comprising:
(i) a pharmaceutical composition comprising C1-INH, and
(ii) instructions for carrying out the method of claim 55.

72. The method of claim 55, wherein determining the Cp comprises:
(i) determining baseline C1-INH functional activity (Cr) in a sample obtained from the patient before C1-INH treatment,
(ii) predefining the desired relative risk reduction h(t),
(iii) determining the corresponding target C1-INH functional activity (Cp) based on a model, and
(iv) determining the C1-INH dosing scheme required to maintain the patient's trough level C1-INH functional activity above the target C1-INH functional activity (Cp).

73. The method of claim 72, wherein the model allows determining Cp based on Cr and relative h(t), wherein Cr is the baseline value determined in step (i) and relative h(t) is the desired relative risk reduction predefined in step (ii).

74. The method of claim 72, wherein the model is
$$Cp = \frac{{e}^{3.4} \times ( log ( relative h ( t ) ) + \frac{- 10.5 \times Cr}{{e}^{3.4} + Cr} )}{- 10.5 - log ( relative h ( t ) ) - \frac{- 10.5 \times Cr}{{e}^{3.4} + Cr}}$$
wherein Cr is the baseline value determined in step (i) and relative h(t) is the desired relative risk reduction predefined in step (ii).

75. A method for adjusting a dosing scheme for C1-INH for the treatment of hereditary angioedema and/or the prevention of hereditary angioedema attacks in an individual patient comprising the following steps:
(i) determining baseline C1-INH functional activity (Cr) in a sample obtained from the patient before C1-INH treatment,
(ii) determining trough C1-INH functional activity in a sample obtained from the patient during ongoing treatment with a standard dose of C1-INH,
(iii) determining the optimal relative risk reduction h(t) based on the patient's treatment response to the treatment of step (ii),
(iv) determining the corresponding target C1-INH functional activity (Cp) based on a model, and
(v) determining the C1-INH dosing scheme required to maintain the patient's trough level C1-INH functional activity above the target C1-INH functional activity based on the trough C1-INH functional activity determined in step (ii).

76. A method of determining a therapeutic C1-INH concentration (Cp) for the treatment of hereditary angioedema and/or the prevention of hereditary angioedema attacks in an individual patient, wherein the Cp is determined using an age-dependent risk-for-an-angioedema-attack model.
